SEQUENCE LISTING (51 sequences)

SEQ ID NO: 1 - protein, 451 aa, synthetic construct
QVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 215 aa, synthetic construct
QIVLTQSPAIMSASLGERVTMTCTASSSVSSNYLHWYQQKPGSAPNLWIYSTSNLASGVPARFSGSGSGTSYSLTISSMEAEDAATYYCHQYLRSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 12 aa, synthetic construct
  VARIANT 3: note = X = S or T
  VARIANT 4: note = X = S or P or T
  VARIANT 5: note = X = Q or N
  VARIANT 7: note = X = F or S
  VARIANT 8: note = X = T or I
  VARIANT 10: note = X = K or T
  VARIANT 12: note = X = G or D or S
TLXXXHXXYXIX

SEQ ID NO: 4-5 - skipped (2 entries)

SEQ ID NO: 6 - protein, 10 aa, synthetic construct
  VARIANT 6: note = X = N or A
  VARIANT 9: note = X = I or V
GFTFSXRWXY

SEQ ID NO: 7 - protein, 19 aa, synthetic construct
  VARIANT 7: note = X = N or A or T
  VARIANT 8: note = X = I or R
  VARIANT 12: note = X = E or Q
GIKTKPXXYATXYADSVKG

SEQ ID NO: 8 - protein, 9 aa, synthetic construct
  VARIANT 1: note = X = L or V
  VARIANT 4: note = X = M or K
  VARIANT 5: note = X = R or K
XTGXXYFDY

SEQ ID NO: 9 - protein, 11 aa, synthetic construct
TLSSQHSTYTI

SEQ ID NO: 10 - protein, 15 aa, synthetic construct
LNSDSSHNKGSGIPD

SEQ ID NO: 11 - protein, 9 aa, synthetic construct
AAYYAYGYV

SEQ ID NO: 12 - protein, 10 aa, synthetic construct
GFTFSARWIY

SEQ ID NO: 13 - protein, 22 aa, synthetic construct
GIKTKPAIYATEYADSVKGRFT

SEQ ID NO: 14 - protein, 9 aa, synthetic construct
LTGMKYFDY

SEQ ID NO: 15 - protein, 12 aa, synthetic construct
TLSSQHSTYTIG

SEQ ID NO: 16 - protein, 11 aa, synthetic construct
LNSDSSHNKGS

SEQ ID NO: 17 - protein, 19 aa, synthetic construct
GIKTKPAIYATEYADSVKG

SEQ ID NO: 18 - protein, 11 aa, synthetic construct
LNSDGSHNKGD

SEQ ID NO: 19 - protein, 9 aa, synthetic construct
GSSYSSGYV

SEQ ID NO: 20 - protein, 10 aa, synthetic construct
GFTFSNRWIY

SEQ ID NO: 21 - protein, 19 aa, synthetic construct
GIKTKPNIYATEYADSVKG

SEQ ID NO: 22 - protein, 9 aa, synthetic construct
LTGMRYFDY

SEQ ID NO: 23 - protein, 11 aa, synthetic construct
LNSSGSHNKGS

SEQ ID NO: 24 - protein, 9 aa, synthetic construct
GYSYYSGYV

SEQ ID NO: 25 - protein, 9 aa, synthetic construct
GSSYSTGYV

SEQ ID NO: 26 - protein, 12 aa, synthetic construct
TLSPQHFTYKID

SEQ ID NO: 27 - protein, 12 aa, synthetic construct
TLSSNHSIYTIS

SEQ ID NO: 28 - protein, 9 aa, synthetic construct
GASYSSGYI

SEQ ID NO: 29 - protein, 9 aa, synthetic construct
VTGMRYFDY

SEQ ID NO: 30 - protein, 12 aa, synthetic construct
TLTTQHSTYTIG

SEQ ID NO: 31 - protein, 19 aa, synthetic construct
GIKTKPTRYATQYADSVKG

SEQ ID NO: 32 - protein, 11 aa, synthetic construct
INSDGSHKKGV

SEQ ID NO: 33 - protein, 11 aa, synthetic construct
LTSHGLHQKGV

SEQ ID NO: 34 - protein, 11 aa, synthetic construct
LNSDSTYITAV

SEQ ID NO: 35 - protein, 11 aa, synthetic construct
RASQSVSSYLA

SEQ ID NO: 36 - protein, 7 aa, synthetic construct
DASNRAT

SEQ ID NO: 37 - protein, 9 aa, synthetic construct
QQRSNWPLT

SEQ ID NO: 38 - protein, 6 aa, synthetic construct
SSSYWG

SEQ ID NO: 39 - protein, 16 aa, synthetic construct
SIYYSGRNYNNPSLKS

SEQ ID NO: 40 - protein, 16 aa, synthetic construct
HYYGSGSSYYYYDLDV

SEQ ID NO: 41 - protein, 11 aa, synthetic construct
RASQSVSNYLA

SEQ ID NO: 42 - protein, 9 aa, synthetic construct
QQRTNWPYT

SEQ ID NO: 43 - protein, 5 aa, synthetic construct
SYAMH

SEQ ID NO: 44 - protein, 17 aa, synthetic construct
VISFDGSNKNYADSVKG

SEQ ID NO: 45 - protein, 12 aa, synthetic construct
EYWGTYYYGMDV

SEQ ID NO: 46 - protein, 9 aa, synthetic construct
QQRSNWPRT

SEQ ID NO: 47 - protein, 6 aa, synthetic construct
STYAMH

SEQ ID NO: 48 - protein, 17 aa, synthetic construct
VISYDGSNKYYADSVKG

SEQ ID NO: 49 - protein, 14 aa, synthetic construct
RESPPIYYYYGMDV

SEQ ID NO: 50 - protein, 17 aa, synthetic construct
VISFDGSTKYYADSVKG

SEQ ID NO: 51 - protein, 12 aa, synthetic construct
EYWGTYYYGTDV

(19) United States
(12) Patent Application Publication
(10) Pub. No.: US 2025/0066485 A1
(43) Pub. Date: Feb. 27, 2025

(54) USE OF INTEGRIN a5b1 INHIBITORS IN THE TREATMENT OF PULMONARY HYPERTENSION AND HEART FAILURE

(71) Applicant: Morphic Therapeutic, Inc., Waltham, MA (US)

(72) Inventors: Adrian S. Ray, Waltham, MA (US); Monica Montesinos, Waltham, MA (US); Hua Wang, Waltham, MA (US); Min Lu, Waltham, MA (US); Fu-Yang Lin, Waltham, MA (US)

(21) Appl. No.: 18/944,958

(22) Filed: Nov. 12, 2024

Related U.S. Application Data
(63) Continuation of application No. PCT/US2023/066955, filed on May 12, 2023.
(60) Provisional application No. 63/499,378, filed on May 1, 2023.
(60) Provisional application No. 63/480,807, filed on Jan. 20, 2023.
(60) Provisional application No. 63/353,306, filed on Jun. 17, 2022.
(60) Provisional application No. 63/341,383, filed on May 12, 2022.

(51) Int. Cl. C07K 16/28; A61K 31/4375; A61K 39/00; A61P 9/12

(52) U.S. Cl. CPC C07K 16/2842; A61K 31/4375; A61P 9/12; A61K 2039/505; C07K 2317/76; C07K 2317/92

(57) ABSTRACT

The present invention provides methods and compositions for treating a lung or heart disease and/or a disease associated with increased expression or activity of integrin α5β1, comprising administering an integrin α5β1, inhibitor to a subject. Methods of treating or preventing pulmonary hypertension, pulmonary arterial hypertension (PAH), right ventricle failure, heart failure and fibrosis are also provided herein.

### CROSS-REFERENCE TO RELATED APPLICATIONS

This application is a Continuation Application of International Application No. PCT/US2023/066955, filed on May 12, 2023, which claims priority to, and the benefit of, U.S. Provisional Application Nos. 63/499,378, filed on May 1, 2023, 63/480,807, filed on Jan. 20, 2023, 63/353,306, filed on Jun. 17, 2022, and 63/341,383, filed on May 12, 2022, the contents of each of which are hereby incorporated by reference in their entireties.

### CROSS REFERERENCE TO SEQUENCE LISTING

The instant application is being filed with an electronically filed Sequence Listing in XML format. The sequence listing file entitled MORF-008US1_SL.XML, was created on Aug. 22, 2023, and is 48,900 bytes in size; the information in electronic format of the Sequence Listing is incorporated herein by reference in its entirety.

### BACKGROUND

Fibronectin (Fn) is an extracellular matrix protein that orchestrates complex cell adhesion and signaling through cell surface integrin receptors (Fibronectin-binding integrins (e.g., a5b1)) during tissue development, remodeling, and disease, such as hypertension and heart failure. Heart failure (HF) is a debilitating disease in which abnormal function of the heart leads to inadequately low perfusion of tissues and organs of the body. Hypertension is a responsible for various deleterious effects and with high morbidity and mortality including heart failure. One form of hypertension is pulmonary arterial hypertension (PAH). PAH is a rare but devastating disease, in which the normally low pulmonary arterial pressure becomes elevated due to vasoconstriction and remodeling of pulmonary vessels. Vasoconstriction and vascular remodeling increases workload on the right side of the heart, causing right heart hypertrophy, fibrosis and ultimately heart failure.

Current treatments include vasodilators targeting Ca channels or endothelin receptors. There is a need for new approaches in the treatment of pulmonary hypertension, PAH, heart failure and related diseases.

### SUMMARY OF THE INVENTION

The present invention is based, in part, on the discovery that integrin signaling could promote cell proliferation and, resistance to apoptosis contributing to vascular remodeling of the pulmonary arterioles and arteries (PAS). In the right ventricle (RV), integrin signaling contributes to maladaptive hypertrophy and fibrosis which can lead to RV failure in pulmonary hypertension. The use of α5β1 inhibitors (e.g., small molecule compounds and antibodies) provided herein reverse PAs vascular remodeling and prevent RV dysfunction.

In one aspect, the present invention provides a method of treating a heart or lung disease in a subject, comprising administering an integrin α5β1 inhibitor. As described herein, a5b1 inhibition not only maintains cardiac output, but also prevents the maladaptation of the RV. Accordingly, the present invention provides methods of treating hypertensive or cardiac disorders, including but not limited to heart failure, RV failure (e.g., RV failure resulting from RV volume overload due to septal defects or valvular regurgitation), RV pressure overload due to other WHO groups of pulmonary hypertension or outflow obstructions such as pulmonary artery stenosis, and RV cardiomyopathies due to infarction, arrythmia, or fibrosis.

The present invention provides a method of treating a disease in which α5β1 function is implicated using integrin α5β1 inhibitors (e.g., small molecule compounds and antibodies). In one aspect, the present invention provides a method of treating a disease associated with increased expression or activity of integrin α5β1, comprising administering an integrin α5β1 inhibitor.

In some embodiments, the disease is characterized by the World Health Organization (WHO) group.

In some embodiments, the disease is pulmonary hypertension, WHO Group 1 pulmonary hypertension or pulmonary arterial hypertension (PAH), WHO Group 2 pulmonary hypertension, WHO Group 3 pulmonary hypertension, WHO Group 4 pulmonary hypertension, and WHO Group 5 pulmonary hypertension.

In some embodiments, the disease is characterized by the World Health Organization (WHO) class system. In some embodiments, the disease is characterized by WHO functional class based on cardiac function. In some embodiments, the disease is pulmonary hypertension, WHO Class I pulmonary hypertension or pulmonary arterial hypertension (PAH), WHO Class II pulmonary hypertension, WHO Class III pulmonary hypertension, WHO Class IV pulmonary hypertension. In some embodiments, the disease is heart failure or right ventricle failure.

In some embodiments, the disease is fibrosis. In some embodiments, the disease is cardiac fibrosis.

In one aspect, the present invention provides a method of treating pulmonary arterial hypertension (PAH) in a subject, comprising administering an integrin α5β1 inhibitor.

In some embodiments, the integrin α5β1 inhibitor is a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof.

In some embodiments, the integrin α5β1 inhibitor is an antibody drug conjugate (ADC).

In some embodiments, the integrin α5β1 inhibitor is an antibody.

In some embodiments, the integrin α5β1 inhibitor is an antibody that specifically binds integrin α5.

In some embodiments, the integrin α5β1 inhibitor is an antibody that specifically binds integrin β1.

In some embodiments, the integrin α5β1 inhibitor is an anti-CD29/β1integrin/ITGB1 monoclonal antibody. In some embodiments, the integrin α5β1 inhibitor antibody is anti CD29/β1integrin/ITGB1 monoclonal antibody OS2966. In some embodiments, the integrin α5β1 inhibitor antibody competes for integrin binding with OS2966.

In some embodiments, the integrin α5β1 inhibitor is an antibody that specifically binds integrin α5β1 heterodimer.

In some embodiments, the antibody is an integrin α5β1 antibody selected from the group consisting of volociximab (M200), PF-04605412 and MINT1526A. In some embodiments, the antibody is volociximab (M200). In some embodiments, the antibody is PF-04605412. In some embodiments, the antibody is MINT1526A.

In some embodiments, the antibody is an integrin α5β1 antibody that competes for integrin binding with an antibody selected from the group consisting of volociximab (M200), P1D6, PF-04605412, MINT1526A, BMA5, BMB5, BMC5, HA5, JBS5, LS-C509074, LS-C24758, 1D9, 22B5, 24C7, 2D2, 3C2.2A8, 3C5, 5B11, MOR04055,MOR04624, P8D4, MOR04974, MOR04977, SG/19, and 18C12.

In some embodiments, the antibody is an integrin α5β1 antibody that competes for integrin binding with the anti-α5β1 antibody clone 339.1.

In some embodiments, the antibody is an integrin α5β1 antibody 3C5 or 5B11. In some embodiments, the antibody is an integrin α5β1 antibody that competes for integrin binding with an antibody selected from 3C5 and 5B11. 3C5 and 5B11 are integrin α5β1 antibodies described in WO2010072740A2, which is hereby incorporated by reference.

In some embodiments, the α5β1 antibody is an anti-Integrin alpha 5 (CD49e) antibody, clone mAb16. In some embodiments, the α5β1 antibody is antibody that competes for integrin binding with anti-integrin alpha 5 (CD49e) antibody, clone mAb16.

In some embodiments, the integrin α5β1 inhibitor is a small molecule compound that binds integrin α5β1.

In some embodiments, the integrin α5β1 inhibitor is aa small molecule compound that specifically binds integrin α5.

In some embodiments, the integrin α5β1 inhibitor is a small molecule compound that specifically binds integrin β1.

In some embodiments, the integrin α5β1 inhibitor is a small molecule compound that specifically binds integrin α5β1 heterodimer.

In some embodiments, the integrin α5β1 inhibitor is a compound of Formula (I) or a pharmaceutically acceptable salt thereof,

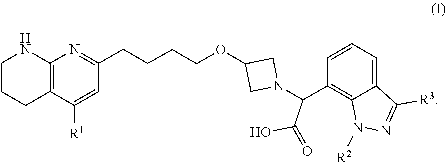

wherein:
R¹ is hydrogen or OMe;
R² is C₁₋₄alkyl optionally substituted with C₁₋₄alkoxy; and
R³ is C₁₋₄alkyl or C₃₋₅cycloalkyl.

In some embodiments, R² is methyl or ethyl.

In some embodiments, R³ is methyl, ethyl, isopropyl, or cyclopropyl.

In some embodiments, R¹ is hydrogen.

In some embodiments, the compound of Formula (I) is selected from:

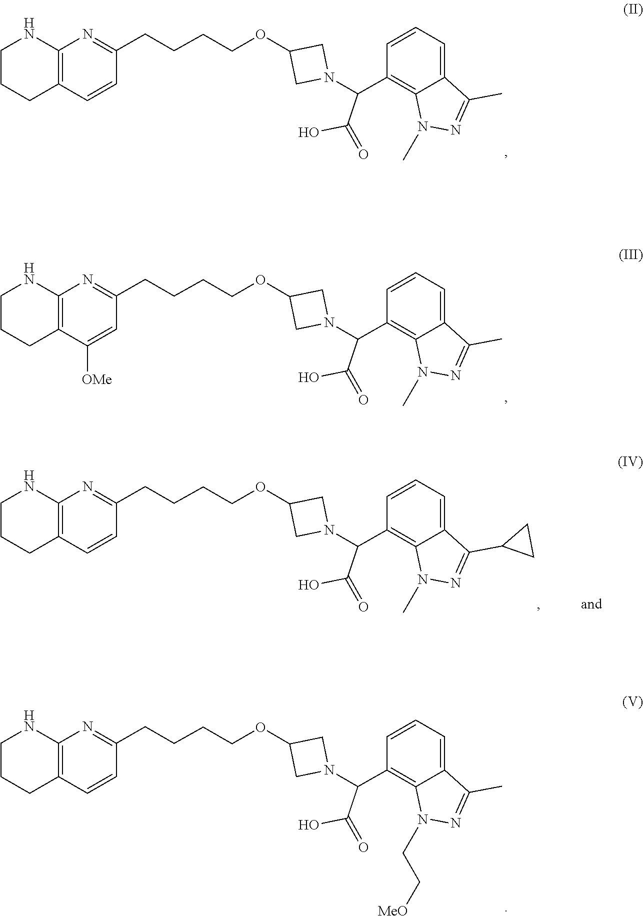 [SMILES: Cc1nn(C)c2c(C(C(=O)O)N3CC(OCCCCc4ccc5c(n4)NCCC5)C3)cccc12 ; COc1cc(CCCCOC2CN(C(C(=O)O)c3cccc4c(C)nn(C)c34)C2)nc2c1CCCN2 ; Cn1nc(C2CC2)c2cccc(C(C(=O)O)N3CC(OCCCCc4ccc5c(n4)NCCC5)C3)c21 ; COCCn1nc(C)c2cccc(C(C(=O)O)N3CC(OCCCCc4ccc5c(n4)NCCC5)C3)c21]

In some embodiments, the integrin α5β1 inhibitor is a compound of Formula (II)

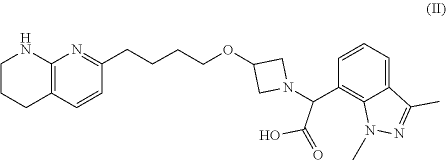 [SMILES: Cc1nn(C)c2c(C(C(=O)O)N3CC(OCCCCc4ccc5c(n4)NCCC5)C3)cccc12]

In some embodiments, the integrin α5β1 inhibitor is administered through inhalation, orally, intravenously, subcutaneously, intranasally, transdermally, intraperitoneally, intramuscularly, or intrapulmonarily. In some embodiments, the integrin α5β1 inhibitor is administered through inhalation.

In some embodiments, the method of treatment described herein, further comprises administering to the subject additional therapies. In some embodiments, the method of treatment comprises administering an integrin α5β1 inhibitor in combination with one or more additional therapies. In some embodiments, the method of treatment further comprises administering to the subject an integrin α5β1 inhibitor and a second therapy. In some embodiments, the method of treatment further comprises administering to the subject an integrin α5β1 inhibitor and two additional therapies.

In some embodiments, the additional therapy is selected from the group consisting of anticoagulants, diuretics, a digitalis glycosides, calcium channel blockers, endothelin receptor antagonists, phosphodiesterase 5 (PDE5) inhibitors, prostanoids, prostanoids receptor agonists, soluble guanylate cyclase stimulators, and/or surgery.

In some embodiments, the additional therapy is oxygen, Warfarin, furosemide, bumetanide, bendroflumethiazide, metolazone, spironolactone, amiloride, Digoxin, nifedipine, diltiazem, nicardipine, amlodipine, ambrisentan, bosentan, macitentan, sildenafil, tadalafil, epoprostenol, iloprost, treprostinil, riociguat, selexipag, surgery, pulmonary endarterectomy, and/or atrial septostomy.

In some embodiments, the additional therapy is macitentan and/or tadalafil.

In some embodiments, the additional therapy is a SGLT2 inhibitor. In some embodiments, the SGLT2 inhibitor is dapagliflozin.

In some embodiments, administering the integrin α5β1 inhibitor reduces proliferation and/or survival of Pulmonary Arterial Smooth Muscle cells (PASMCs), pulmonary artery, right ventricle fibroblasts (RVFbs), vascular fibroblasts, adventitial fibroblasts, cardiomyocytes, and/or endothelial cells.

In some embodiments, the method comprises administering the integrin α5β1 inhibitor to modulate the level of a biomarker in the subject. In some embodiments, the biomarker is N-terminal fragment (NT) of pro-BNP (NT-proBNP), TNFα, IFNγ, IL-6, IL-8,and IL-10. In some embodiments, the biomarker is brain natriuretic peptide (BNP) or N-terminal fragment (NT) of pro-BNP (NT-proBNP).

In some embodiments, the methods of treatment described herein comprises administering the integrin α5β1 inhibitor at a dose of 1 mg to 1000 mg.

In some embodiments, the integrin α5β1 inhibitor is administered daily. In some embodiments, the integrin α5β1 inhibitor is administered two times a day.

In some embodiments, the present invention provides an integrin α5β1 inhibitor for use in treating pulmonary hypertension in a subject in need of treatment thereof, comprising administering the integrin α5β1 inhibitor and a pharmaceutical excipient to the subject.

In some embodiments, the present invention provides an integrin α5β1 inhibitor for use in treating pulmonary arterial hypertension (PAH) in a subject in need of treatment thereof, comprising administering the integrin α5β1 inhibitor and a pharmaceutical excipient to the subject.

In some embodiments, the present invention provides an integrin α5β1 inhibitor for use in treating right ventricle failure in a subject in need of treatment thereof, comprising administering the integrin α5β1 inhibitor and a pharmaceutical excipient to the subject.

### DEFINITIONS

In order for the present invention to be more readily understood, certain terms are first defined below. Additional definitions for the following terms and other terms are set forth throughout the specification.

“Active agent” and “therapeutic agent” means a molecule (e.g., a small molecule compound, peptides, an antibody or antibody fragment, etc.) that exerts a preventive or therapeutic effect on a disease or disease condition. Active agent may refer not only to a single active agent but also to a combination of two or more different active agents.

“Alleviate” and “ameliorate” means a process by which the severity of a sign or symptom of a disorder is decreased. Importantly, a sign or symptom can be alleviated without being eliminated. Therapeutically effective dosages are expected to decrease the severity of, and so alleviate and ameliorate, a sign or symptom of disease.

As used herein, the term “affinity” refers to the characteristics of a binding interaction between a binding moiety (e.g., integrin α5β1 inhibitor) and a target (e.g., α5β, αvβ1) and that indicates the strength of the binding interaction. In some embodiments, the measure of affinity is expressed as a dissociation constant (K_{D}). In some embodiments, a binding moiety has a high affinity for a target (e.g., a K_{D} of less than about 10⁻⁷ M, less than about 10⁻⁸ M, or less than about 10⁻⁹ M). In some embodiments, a binding moiety has a low affinity for a target (e.g., a K_{D} of higher than about 10⁻⁷ M, higher than about 10⁻⁶ M, higher than about 10⁻⁵ M, or higher than about 10⁻⁴ M).

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. An antibody typically includes three complementarity determining regions (abbr. CDRs) in the light chain of the immunoglobulin and three complementarity determining regions (CDRs) in the heavy chain of the immunoglobulin. The three CDRs in the light chain of the immunoglobulin are called, from the N-terminal side, CDR1, CDR2 and CDR3, respectively. The three CDRs in the heavy chain of the immunoglobulin are also called, from the N-terminal side, CDR1, CDR2 and CDR3, respectively. A “CDR” may be identified in accordance with the definitions of the Kabat, Chothia, the accumulation of both Kabat and Chothia, AbM, contact, and/or conformational definitions or any method of CDR determination well known in the art. The term “antibody” encompasses antigen-binding fragments of antibodies (e.g., single chain antibodies, Fab, F(ab′)2, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda.

“Combination therapy” and “co-therapy” means the administration of a first active agent and at least a second, different active agent as part of a specific treatment regimen intended to provide the beneficial effect from the co-action of the at least two active agents. The beneficial effect of the combination may include, but is not limited to, pharmacokinetic or pharmacodynamic co-action resulting from the combination of therapeutic agents. Administration of therapeutic agents in combination may be carried out over a defined time period (e.g., minutes, hours, days or weeks depending upon the combination selected). In some embodiments, the integrin α5β1 inhibitor is administered through inhalation. Combination therapy is not intended to encompass the administration of two or more different therapeutic agents as part of separate monotherapy regimens that incidentally and arbitrarily results in a combination therapy of the invention. Combination therapy includes administration of at least two different therapeutic agents in a sequential manner, wherein each therapeutic agent is administered at a different time, as well as administration of at least two different therapeutic agents in a substantially simultaneous manner. Substantially simultaneous administration may be accomplished, for example, by administering to the subject a single capsule having a fixed ratio of each therapeutic agent or in separate capsules for each of therapeutic agents. Sequential or substantially simultaneous administration of each therapeutic agent may be affected by any appropriate route, including, but not limited to, oral routes, intravenous routes, intramuscular routes, and direct absorption through mucous membrane tissues. The two different therapeutic agents may be administered by the same route or by different routes. For example, a first therapeutic agent of the combination selected may be administered by intravenous injection while the second therapeutic agent of the combination may be administered orally. Alternatively, for example, all therapeutic agents may be administered by inhalation, orally or all therapeutic agents may be administered by intravenous injection. The sequence in which therapeutic agents are administered is not critical, unless otherwise stated.

Combination therapy also includes the administration of the different therapeutic agents as described above in further combination with other biologically active ingredients and non-drug therapies (e.g., surgery or physical therapy). Where a combination therapy comprises a non-drug treatment, the non-drug treatment may be conducted at any suitable time so long as a beneficial effect from the co-action of the combination of therapeutic agents and non-drug treatment is achieved. For example, in appropriate cases, the beneficial effect is still achieved when the non-drug treatment is temporally removed from the administration of therapeutic agents, perhaps by days or even weeks.

“Compound” means a molecule and encompasses not only the specified molecular entity but, if the compound is an active agent or drug, also its pharmaceutically acceptable, pharmacologically active analogs, including, but not limited to, active metabolites, amides, conjugates, esters, hydrates, polymorphs, prodrugs, salts, solvates, and other such derivatives, analogs, including deuterated analogs and analogs containing radioactive atoms or other labeling moieties, and related compounds. In some embodiments, a compound is a small molecule compound.

“Dosage form” means any form of a pharmaceutical composition for administration to a subject (typically a human or animal of veterinary interest suffering from a disease or condition to be treated). “Dose” refers to an amount of active agent. “Unit dosage form” refers to a dosage form that contains a fixed amount of active agent. A single tablet or capsule is a unit dosage form. Multiple unit dosage forms can be administered to provide a therapeutically effective dose. A dosage form can include a combination of dosage forms.

“Effective amount” and “therapeutically effective amount” refers to a nontoxic but sufficient amount of an active agent to achieve a desired therapeutic effect.

“Integrin inhibitor” or “Integrin α5β1 inhibitor” or “VLA5 inhibitor” refers to a molecule that can bind to integrin alpha 5 (ITGA5) and has α5β1 integrin and/or ITGA5 inhibitory activity. In some embodiments, inhibiting activity comprises inhibition of binding of α5β1 integrin and/or ITGA5 to smooth muscle cells, fibroblasts, stellate cells, myofibroblasts, pericytes and/or other cells of mesenchymal origin. In some embodiments, inhibiting activity comprises inhibition of migration of smooth muscle cells, fibroblasts, stellate cells, myofibroblasts, pericytes and/or other cells of mesenchymal origin. In some embodiments, inhibiting activity comprises inhibition of differentiation of smooth muscle cells, fibroblasts, stellate cells, myofibroblasts, pericytes and/or other cells of mesenchymal origin. In some embodiments, inhibiting activity comprises inhibition of extracellular matrix synthesis and/or deposition.

As used herein, “K_{D}” refers to a dissociation constant, which is obtained from the ratio of K_{d} to Kₐ (i.e., K_{d}/Kₐ) and is expressed as a molar concentration (M). K_{D} values can be determined using methods well established in the art, e.g., by using surface plasmon resonance, or using a biosensor system such as a Biacore® system.

As used herein, “Pulmonary arterial hypertension (PAH)” refers to a rare disease, in which the normally low pulmonary artery pressure becomes elevated due to vaso-constriction and to the remodeling of pulmonary vessels. This in turn increases workload on the right side of the heart, causing right heart hypertrophy, fibrosis and ultimately heart failure.

As used herein a “peptide” refers to a peptide or polypeptide that comprise multiple amino acids. The terms “peptide” and “polypeptide” are used interchangeably. The amino acid sequence or variant thereof can be part of a larger peptide, i.e. of a peptide that has been N terminally and/or C-terminally extended by a one or more additional amino acids. The amino acid sequence or variant thereof of a peptide of the invention may also be N-terminally and/or C-terminally modified, preferably by comprising an N- and/or C-terminal clongating group. Alternatively, said amino acid sequence or a variant thereof is N- and/or C-terminally extended.

“Pharmaceutically acceptable” means not biologically undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug Administration.

“Pharmaceutically acceptable salts” mean derivatives of an active agent produced by making acid or base salts thereof. Examples of pharmaceutically acceptable salts include, but are not limited to, mineral or organic acid salts of basic residues such as amines, alkali or organic salts of acidic residues such as carboxylic acids, and the like. Pharmaceutically acceptable salts include the conventional non-toxic salts or the quaternary ammonium salts of the parent compound formed, for example, from non-toxic inorganic or organic acids. Pharmaceutically acceptable salts include those formed when an acidic proton present in the parent compound either is replaced by a metal ion, e.g., an alkali metal ion, an alkaline earth ion, or on aluminum ion; or coordinates with an organic base such as ethanolamine, diethanolamine, triethanolamine, tromethamine, N-methylglucamine, and the like. Pharmaceutically acceptable salts include solvent addition forms (solvates) or crystal forms (polymorphs) as defined herein, of the same salt.

“Pharmacologically active” (or “active”) as in a “pharmacologically active” derivative or analog, refers to a derivative or analog having the same type of pharmacological activity as the parent compound of approximately equivalent in degree.

“Preventing” and “prevent” means avoiding the onset of a clinically evident disease progression altogether or slowing the onset of a pre-clinically evident stage of a disease in individuals at risk. Prevention includes prophylactic treatment of those at risk of developing a disease.

As used herein, “selective binding”, “selectively binds” “specific binding”, or “specifically binds” refers, with respect to a binding moiety and a target, preferential association of a binding moiety to a target and not to an entity that is not the target. A certain degree of non-specific binding may occur between a binding moiety and a non-target. In some embodiments, a binding moiety selectively binds a target if binding between the binding moiety and the target is greater than 2-fold, greater than 5-fold, greater than 10-fold, or greater than 100-fold as compared with binding of the binding moiety and a non-target. In some embodiments, a binding moiety selectively binds a target if the binding affinity is less than about 10⁻⁵ M, less than about 10⁻⁶ M, less than about 10⁻⁷ M, less than about 10⁻⁸ M, or less than about 10⁻⁹ M

“Sign” means an indication of disease and includes conditions that can be observed by a doctor, nurse, or other health care professional.

“Small molecule” as used herein refers to molecules, whether naturally-occurring or artificially created (e.g., via chemical synthesis) that have a relatively low molecular weight. Preferred small molecules are biologically active in that they produce a local or systemic effect in animals, preferably mammals, more preferably humans. In certain preferred embodiments, the small molecule is a drug and the small molecule is referred to as “drug molecule” or “drug” or “therapeutic agent”. The small molecule can have a MW less than or equal to about 5 kDa. In other embodiments, the drug molecule has a MW less than or equal to about 1.5 kDa.

As used herein, the term “subject”, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

“Subject in need thereof” refers to a human or other mammal suitable for treatment with an active agent. A subject in need thereof may have a disease or be at an increased risk, relative to the general population, of developing a disease.

“Symptom” means a sign or other indication of disease, illness, or injury. Symptoms may be felt or noticed by the individual experiencing them or by others, including by non-health-care professionals.

As used herein, the term “therapeutically effective amount” refers to an amount of a therapeutic molecule (e.g., an integrin a5b1 inhibitor described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

“Treating” and “treat” describes the management and care of a patient for the purpose of combating a disease, condition, or disorder and includes the administration of an active agent to alleviate the symptoms or complications of a disease, condition or disorder, or to eliminate the disease, condition or disorder.

“Pulmonary hypertension (PH)” includes diseases which share the defining element of a mean pulmonary arterial pressure ≥25 mm Hg. PH has been classified and divided into 5 groups and 5 classes characterized by the World Health Organization (WHO). In some embodiements, pulmonary hypertension is WHO Functional Class I pulmonary hypertension, WHO Functional Class II pulmonary hypertension, WHO Functional Class III pulmonary hypertension, WHO Functional Class IV pulmonary hypertension or pulmonary arterial hypertension (PAH). In some embodiments, the disease is pulmonary hypertension, WHO Group 1 pulmonary hypertension or pulmonary arterial hypertension (PAH), WHO Group 2 pulmonary hypertension, WHO Group 3 pulmonary hypertension, WHO Group 4 pulmonary hypertension, and WHO Group 5 pulmonary hypertension.

### BRIEF DESCRIPTION OF THE DRAWINGS

Drawings are for illustration purposes only, not for limitation.

FIG. 1 shows RNA expression of human integrins expressed in PAH-patient derived pulmonary arterial smooth muscle cells.

FIG. 2A-2B show exemplary proliferation of hPASMCs treated with anti-integrin a5b1 antibodies, M200 (FIG. 2A) and P1D6 (FIG. 2B).

FIG. 3 shows exemplary proliferation of hPASMCs on plastic and fibronectin-coated plates.

FIG. 4A-4C show exemplary western blot analysis measuring expression levels of p-FAK (FIG. 4B) and PCNA (FIG. 4B) of hPASMCs at 72 hours following exposure to treated with integrin α5β1 inhibitors (P1D6) on fibronectin Fb-coated plates.

FIG. 5A-5C show exemplary proliferation (Ki67) and apoptosis (annexin V) of PAH-patient derived pulmonary arterial smooth muscle cells (PAH-PASMCs) treated with integrin α5β1 inhibitors (MRT, SMi, P1D6, or M200) on Fibronectin (Fb) coated plates.

FIG. 6A-6B show exemplary western blot analysis measuring expression levels of ITGα5, ITGαV, ITGβ1, p-FAK, survivin, p-ERK/ERK, MCM2, PLK1, and PCNA in hPASMCs following exposure to SMi at 0.25 μM, 1 μM, and 4 μM on Fb-coated plates.

FIG. 7A-7C show exemplary proliferation (Ki67) and apoptosis (annexin V) of hPASMCs treated with integrin α5β1 inhibitors (SMi) on Fb-coated plates.

FIG. 8 shows exemplary histology staining of EVG, αSMA, PCNA, and C3C demonstrating vascular remodeling in Sugen/Hypoxia (SuHx)-induced PAH rats.

FIG. 9A-9F shows exemplary results demonstrating efficacy of SMi in SuHx rats following treatment with SMi and/or endothelin receptor antagonist and PDE5. Cardiac output (FIG. 9A), stroke volume (FIG. 9B), media cell wall thickness (FIG. 9C), vascular remodeling (FIG. 9D) proliferation (FIG. 9E) and apoptosis (FIG. 9F) were measured. Dotted lines indicate the disease window.

FIG. 10A-10C shows exemplary results demonstrating reduced cardiac hypertrophy and cardiac fibrosis in SuHx rats following treatment with SMi and/or SoCs (Macitentan (Maci) and Tadalafil (Tada)).

FIG. 11 shows exemplary histology staining of EVG, αSMA, PCNA, and C3C demonstrating vascular remodeling in monocrotaline (MCT) rat pulmonary hypertension model.

FIG. 12A-12D show exemplary results of treatment with SMi in MCT rats. Occlusion score (FIG. 12A), vascular remodeling (FIG. 12B), apoptosis (FIG. 12C) and proliferation (FIG. 12D) were measured.

FIG. 13 shows exemplary pharmacokinetics of SMi and MRT treatment in MCT rats.

FIG. 14A-14E show exemplary improved vascular and cardiac function demonstrated by histology staining of EVG (FIG. 14A), media cell wall thickness (FIG. 14B), mean pulmonary arterial pressure (FIG. 14C), cardiac output (FIG. 14D) and right ventricular fractional area change (FIG. 14E) following SMi and MRT treatment in MCT rats.

FIG. 15A-15C show exemplary histology results demonstrating improved cardiomyocytes hypertrophy and right ventricle (RV) fibrosis in MCT rats treated with SMi and/or SOCs (e.g., Macitentan (Maci) and Tadalafil (Tada)).

FIG. 16A-16D show study design and exemplary histology results demonstrating improved hypertrophy and right ventricle (RV) fibrosis in Pulmonary Arterial banding (PAB) rat model treated with SMi.

FIG. 17 shows representative echocardiograph images of PAB rats treated with SMi.

FIG. 18A-18H demonstrate integrin α5β1 inhibition with SMi showed a direct improvement of cardiac function over time compared to vehicle control in the RV PAB model. Cardiac output (FIG. 18A), stroke volume (FIG. 18B), TAPSE (FIG. 18C), S Wave (FIG. 18D), RVFAC (FIG. 18E), RVEDD (FIG. 18F), CI (FIG. 18G), PG (FIG. 18H)

FIG. 19A-19E show exemplary cardiac parameters of Right Heart Catheterization in PAB rats treated with SMi.

FIG. 20A-20D show exemplary expression of fibronectin binding integrins in PAH patient samples.

FIG. 21A-21C show dose-dependent reduction PE-induced adult rat cardiomyocytes hypertrophy following exposure with SMi treatment on.

FIG. 22A-22B show exemplary results of a5b1 inhibition on Human PAH-RVFbs proliferation and activation.

FIG. 23A-23C show exemplary exemplary results demonstrating improved hemodynamics and vascular remodeling in MCT rats with established PAH following treatment with SMi alone or in combination with SoCs (Macitentan (Maci) and Tadalafil (Tada)) in MCT rat pulmonary hypertension model.

FIG. 24A-24G shows exemplary study design (FIG. 24A) and results demonstrating efficacy of integrin α5β1 inhibitors (SMi and anti-α5β1 antibody 339.1) in SuHx mice. Cardiac output (FIG. 24B), stroke volume (FIG. 24C), Right ventricular systolic pressure (FIG. 24D), mean pulmonary arterial pressure (FIG. 24E), Fulton index (FIG. 24F) and vascular remodeling (FIG. 24G) were measured. Dotted lines indicate the disease window.

FIG. 25A-25B show exemplary protein levels of a5b1 integrin in human (FIG. 25A) and mouse tissues (FIG. 25B).

### DETAILED DESCRIPTION

The present invention provides methods and compositions for treating a disease associated with increased expression or activity of integrin α5β1, comprising administering an integrin α5β1 inhibitor. In one aspect, the present invention provides a method of treating a heart or lung disease in a subject, comprising administering an integrin α5β1 inhibitor. In some embodiments, the disease is characterized by the World Health Organization (WHO) group.

In some embodiments, the disease is pulmonary hypertension, WHO Group 1 pulmonary hypertension or pulmonary arterial hypertension (PAH), WHO Group 2 pulmonary hypertension, WHO Group 3 pulmonary hypertension, WHO Group 4 pulmonary hypertension, and WHO Group 5 pulmonary hypertension.

In some embodiments, the disease is characterized by the World Health Organization (WHO) class system. In some embodiments, the disease is characterized by WHO functional class based on cardiac function. In some embodiments, the disease is pulmonary hypertension, WHO Class I pulmonary hypertension or pulmonary arterial hypertension (PAH), WHO Class II pulmonary hypertension, WHO Class III pulmonary hypertension, WHO Class IV pulmonary hypertension. In some embodiments, the disease associated with increased expression or activity of integrin α5β1 is heart failure or right ventricle failure.

Due to the limitations of current treatments for pulmonary hypertension, there remains a significant interest in and need for additional or alternative therapies for treating, stabilizing, preventing, and/or delaying pulmonary hypertension. Various processes have been developed in order to obtain more efficient and/or less toxic drugs for the treatment of pulmonary hypertension. However, these processes still present serious side effects, and the resulting drugs often exhibit short half-life and low bioavailability.

The present invention provides, among other things, methods, and compositions for treating a disease associated with increased expression or activity of integrin α5β1. Many normal physiological and disease processes require cells to contact other cells and/or extracellular matrix. Cell-matrix and cell-cell adhesion is mediated through several families of proteins including integrins, selectins, cadherins, and immunoglobulins, and facilitates a variety of normal cellular functions such as proliferation, migration, differentiation, or survival. Cell adhesion is also key to a range of pathologies, and so pharmacological disruption of cell adhesion interactions can provide a mechanism for therapeutic intervention. Members of the integrin superfamily adhesion molecules play an important role in acute and chronic disease states such as cancer, inflammatory diseases, stroke, and neurodegenerative disorders. Thus, integrins represent a complex biological area.

The integrin superfamily of cell surface receptors is formed from a number of structurally and functionally related surface glycoproteins, with each receptor existing as a 20 heterodimer of non-covalently linked α and β subunits. At least 18 different α and 8 β subunits have been identified in mammals, which are known to form more than 24 different receptors. Each integrin interacts specifically with defined extracellular ligands, including extracellular matrix proteins such as, fibronectin, vitronectin, collagen and cell surface molecules such as VCAM, ICAM and PECAM, via linear 25 adhesion motifs.

The integrin α5β1 (a5b1 or alpha5 beta1) is composed of an α5 (a5 or alpha5) and β1 (b1 or beta1) subunit. The α5 subunit forms a specific dimer with the beta1 subunit and is widely expressed in most tissues. Integrin a5b1 almost exclusively mediates cell adhesion through an interaction with fibronectin, binding via the short arginine-glycine-30 aspartate (RGD) adhesion motif. Endothelial cells and platelets can however bind to fibrin via a5b1. The a5b1 interaction with fibronectin plays an important role in physiopathological angiogenesis and vascular integrity. Although endothelial cells express a variety of integrins, a5b1 is important for survival of endothelial cells on provisional matrix in vitro, suppressing apoptosis and promoting proliferation. a5b1 expression is upregulated in tumor vasculature and pulmonary hypertension patients. Consistent with a key functional role for the receptor-ligand pairing, the a5b1 ligand fibronectin is also upregulated in tumor tissue and during wound-healing.

As demonstrated herein, inhibition of integrin α5β1 by blocking the activity of α5β1 or inhibition of α5β1 fibronectin binding is effective in preventing and treating pulmonary hypertension, PAH, heart failure and right ventricle failure. The present invention provides a variety of compounds (e.g., small molecule compounds and antibodies) that inhibit that interaction. The compounds are referred to generically herein as “integrin a5b1 inhibitors”.

Various aspects of the invention are described in detail in the following sections. The use of sections is not meant to limit the invention. Each section can apply to any aspect of the invention. In this application, the use of “or” means “and/or” unless stated otherwise.

### Pulmonary Hypertension

Pulmonary hypertension (PH) is a syndrome characterized by increased pulmonary artery pressure. PH is defined hemodynamically as a systolic pulmonary artery pressure greater than 30 mm Hg or evaluation of mean pulmonary artery pressure greater than 25 mm Hg. See Zaiman et al., Am. J. Respir. Cell Mol. Biol. 33:425-31 (2005). Further, PH, as a result of the increased pressure, damages both the large and small pulmonary arteries. The walls of the smallest blood vessels thicken and are no longer able to transfer oxygen and carbon dioxide normally between the blood and the lungs. In time, pulmonary hypertension leads to thickening of the pulmonary arteries and narrowing of the passageways through which blood flows. Once pulmonary hypertension develops, the right side of the heart works harder to compensate; however, the increased effort causes it to become enlarged and thickened. Proliferation of smooth muscle and endothelial cells which normally exist in a quiescent state leads to remodeling of the vessels with obliteration of the lumen of the pulmonary vasculature. This causes a progressive rise in pulmonary pressures as blood is pumped through decreased lumen area. The enlarged right ventricle places a person at risk for pulmonary embolism because blood tends to pool in the ventricle and in the legs. If clots form in the pooled blood, they may eventually travel and lodge in the lungs. The progressive rise in pressure also places an additional workload on the right ventricle which eventually fails and leads to premature death in these patients.

Various pathologic changes occur in pulmonary arteries as a result of PH. Persistent vasoconstriction and structural remodeling of the pulmonary vessels are cardinal features of PH. Pulmonary vascular smooth muscle cells undergo a phenotypic switch from contractile normal phenotype to a synthetic phenotype leading to cell growth and matrix deposition. Histological examination of tissue samples from patients with pulmonary hypertension shows intimal thickening, as well as smooth muscle cell hypertrophy, especially for those vessels <100 m diameter. Further, abnormal smooth muscle cells often overexpress endothelin and serotonin transporters, which likely play a role in the development of PH.

The most common symptom of pulmonary hypertension initially is shortness of breath upon exertion. Some people feel light-headed or fatigued upon exertion, and an angina-like chest pain is common. Because body tissues are not receiving enough oxygen, general weakness is another problem. Other symptoms, such as coughing and wheezing, may be caused by an underlying lung disease. Edema, particularly of the legs, may occur because fluid may leak out of the veins and into the tissues, signaling that cor pulmonale has developed. Some people with pulmonary hypertension have connective tissue disorders, especially scleroderma. When people have both conditions, pulmonary hypertension and connective tissue disorders, Raynaud's phenomenon often develops before symptoms of pulmonary hypertension appear, sometimes as long as years earlier.

Treatment of some types of pulmonary hypertension is often directed at the underlying lung disease. Currently, the treatment options available for those suffering from PH target cellular dysfunction that leads to constriction of the vasculature. Therapies such as prostanoids, phosphodiesterase-5 inhibitors and endothelin receptor antagonists primarily work by causing dilation of the pulmonary vessels. Vasodilators, such as calcium channel blockers, nitric oxide, and prostacyclin, are often helpful for pulmonary hypertension associated with scleroderma, chronic liver disease, and HIV infection. In contrast, these drugs have not been proven effective for people with pulmonary hypertension due to an underlying lung disease. For most people with pulmonary hypertension due to an unknown cause, vasodilators, such as prostacyclin, drastically reduce blood pressure in the pulmonary arteries. Prostacyclin given intravenously through a catheter surgically implanted in the skin improves the quality of life, increases survival, and reduces the urgency of lung transplantation. Unfortunately, many patients respond poorly to these therapies or stop responding to them over time. The only remaining option at that point in time is a single or double lung transplantation to treat PH. Although there is some evidence that available therapies have secondary effects on vascular remodeling, there are currently no therapies that target abnormal cell proliferation in PAH.

In some embodiments, the pulmonary hypertension is pulmonary venous hypertension (PVH). In some embodiments, the PVH is due to left heart failure. In some embodiments, the pulmonary hypertension is pulmonary hypertension associated with disorders of the respiratory system and/or hypoxia. In some embodiments, the pulmonary hypertension is pulmonary hypertension due to chronic thrombotic and/or embolic disease. In some embodiments, the pulmonary hypertension is miscellaneous pulmonary hypertension. In some embodiments, the miscellaneous pulmonary hypertension is associated with sarcoidosis, eosinophilic granuloma, histicytosis X, lymphangiolomyiomatosis, or compression of pulmonary vessels (e.g., adenopath, tumor, or fibrosing medianstinitis). In some embodiments, the pulmonary hypertension is associated with chronic obstructive pulmonary disease (COPD). In some embodiments, the pulmonary hypertension is associated with pulmonary fibrosis. In some embodiments, the pulmonary hypertension is associated with cardiac fibrosis. In some embodiments, the pulmonary hypertension is early-stage pulmonary hypertension or advanced pulmonary hypertension.

In some embodiments, the subject suffers from pulmonary venous hypertension (PVH). In some embodiments, the PVH is due to left heart failure. In some embodiments, the subject suffers from a disorder of the respiratory system and/or hypoxia. In some embodiments, the subject suffers from a chronic thrombotic and/or embolic disease. In some embodiments, the subject suffers from sarcoidosis, cosinophilic granuloma, histicytosis X, lymphangiolomyiomatosis, or compression of pulmonary vessels (e.g., due to an adenopathy, a tumor, or fibrosing medianstinitis). In some embodiments, the subject suffers from chronic obstructive pulmonary disease (COPD). In some embodiments, the subject suffers from pulmonary fibrosis. In some embodiments, the subject suffers from cardiac fibrosis. In some embodiments, the subject suffers from early-stage pulmonary hypertension or advanced pulmonary hypertension.

In some embodiments, one or more symptoms of the pulmonary hypertension are ameliorated. In some embodiments, the pulmonary hypertension is delayed. In some embodiments, the pulmonary hypertension is prevented. In some embodiments, the methods of treatment provided herein reduce pulmonary pressure. In some embodiments, the methods of treatment provided herein inhibit and/or reduce abnormal cell proliferation in the pulmonary artery.

In some embodiments, the pulmonary hypertension is characterized by the World Health Organization (WHO) group.

In some embodiments, the pulmonary hypertension is WHO Group 1 pulmonary hypertension or pulmonary arterial hypertension (PAH), WHO Group 2 pulmonary hypertension, WHO Group 3 pulmonary hypertension, WHO Group 4 pulmonary hypertension, and WHO Group 5 pulmonary hypertension.

In some embodiments, the pulmonary hypertension is characterized by the World Health Organization (WHO) class system. In some embodiments, the pulmonary hypertension is characterized by WHO functional class based on cardiac function. In some embodiments, the pulmonary hypertension is characterized as WHO Class I pulmonary hypertension or pulmonary arterial hypertension (PAH), WHO Class II pulmonary hypertension, WHO Class III pulmonary hypertension, WHO Class IV pulmonary hypertension.

In some embodiments, the subject suffers from pulmonary hypertension characterized by the World Health Organization (WHO) group.

In some embodiments, the subject suffers from WHO Group 1 pulmonary hypertension or pulmonary arterial hypertension (PAH), WHO Group 2 pulmonary hypertension, WHO Group 3 pulmonary hypertension, WHO Group 4 pulmonary hypertension, or WHO Group 5 pulmonary hypertension.

In some embodiments, the subject suffers from a disease characterized by the World Health Organization (WHO) class system. In some embodiments, the subject suffers from a disease characterized by WHO functional class based on cardiac function. In some embodiments, the subject suffers from pulmonary hypertension classified by WHO Class I pulmonary hypertension or pulmonary arterial hypertension (PAH), WHO Class II pulmonary hypertension, WHO Class III pulmonary hypertension, WHO Class IV pulmonary hypertension.

In some embodiments, the pulmonary hypertension is associated with pulmonary capillary hemangiomatosis.

### Pulmonary Arterial Hypertension (PAH)

In one aspect, the present invention provides a method of treating Pulmonary arterial hypertension (PAH), comprising administering an integrin α5β1 inhibitor (e.g., small molecule compounds and antibodies disclosed herein). PAH is characterized by a progressive increase in pulmonary vascular resistance leading to right ventricular overload and eventually cardiac failure. PAH results in progressive obstruction and decreased compliance of pulmonary arteries (PA), leading to right ventricular (RV) failure and premature death. Like cancer cells, PA smooth muscle cells (PASMCs) and endothelial cells (PAECs) exhibit exaggerated proliferation and resistance to apoptosis in response to increased PA stiffness caused by extracellular matrix (ECM) remodeling. Integrins signaling could promote PAH-PASMCs and PAH-PAECs proliferation and resistance to apoptosis contributing to PAs vascular remodeling, while in the RV, maladaptive hypertrophy, and fibrosis, leading to RV failure in PAH. The present invention is based, in part, on the discovery that a5b1 integrin inhibition could reverse PAs vascular remodeling and prevent RV dysfunction in PAH.

PAH is a chronic disorder that involves all layers of the pulmonary vessels. Vasoconstriction, structural changes in the pulmonary vessel wall (vascular remodeling) and thrombosis contribute to the increased pulmonary vascular resistance in PAH. Structural and functional changes of the endothelium lead to endothelial dysfunction. Increased vasoconstrictive factors (e.g., endothelin) and decreased vasodilation capacity (e.g., less prostacyclin) result in vasoconstriction and increased pulmonary vascular resistance. Current treatments that seek to address vasoconstriction may slow the progression of PAH or ameliorate the clinical symptoms for a limited time, but they have not proven to substantially reduce overall PAH morbidity and mortality rates. Underlying structural changes to the pulmonary vessels—vascular remodeling—are not affected by these treatments.

Vascular remodeling that occurs in PAH is characterized by proliferative and obstructive changes involving many cell types, including endothelial cells, smooth muscle cells and fibroblasts. Vascular remodeling can manifest itself, for example, as medial thickening of pulmonary vessels due to smooth muscle cell hyperplasia and hypertrophy, formation of a neointima made of smooth muscle cells and/or myofibroblasts, and/or formation of plexiform lesions, which consist of localized proliferations of endothelial cells, smooth muscle cells, lymphocytes, and mast cells. Vascular remodeling results in obstruction of the vessel lumen leading to pulmonary hypertension. There is a need for therapies that address the proliferative aspect of PAH.

In some embodiments, the pulmonary hypertension is pulmonary arterial hypertension (PAH). In some variations, the PAH is idiopathic PAH. In some variations, the PAH is familial PAH. In some variations, the PAH is associated with persistent pulmonary hypertension of a newborn. In some variations, the PAH is associated with pulmonary veno-occlusive disease.

In some embodiments, the pulmonary hypertension is associated with lung diseases. In some embodiments, the lung disease is Idiopathic pulmonary fibrosis (IPF) or interstitial pneumonia (IIP). IPF is a type of idiopathic interstitial pneumonia (IIP), which in turn is a type of interstitial lung disease (also known as diffuse parenchymal lung disease (DPLD)). Interstitial lung disease concerns alveolar epithelium, pulmonary capillary endothelium, basement membrane, perivascular and perilymphatic tissues. Other forms of idiopathic interstitial pneumonias include non-specific interstitial pneumonia (NSIP), desquamative interstitial pneumonia (DIP) and acute interstitial pneumonia (AIP). Examples of known causes of interstitial lung disease include sarcoidosis, hypersensitivity pneumonitis, pulmonary Langerhans cell histiocytosis, asbestosis and collagen vascular diseases such as scleroderma and rheumatoid arthritis.

Accordingly, in some embodiments, the subject suffers from a lung disease such as Idiopathic pulmonary fibrosis (IPF) or interstitial pneumonia (IIP). In some embodiments, the subject suffers from idiopathic interstitial pneumonia (IIP), diffuse parenchymal lung disease (DPLD), non-specific interstitial pneumonia (NSIP), desquamative interstitial pneumonia (DIP), or acute interstitial pneumonia (AIP). In some embodiments, the subject suffers from sarcoidosis, hypersensitivity pneumonitis, pulmonary Langerhans cell histiocytosis, asbestosis or a collagen vascular disease such as scleroderma or rheumatoid arthritis.

Pulmonary fibrosis is the formation or development of excess fibrous connective tissue in the lungs.

### Heart Failure (HF)

In one aspect, the present invention provides a method of treating heart failure (HF), comprising administering an integrin α5β1 inhibitor (e.g., small molecule compounds and antibodies disclosed herein). Heart failure refers to any condition characterized by the heart's inability to pump an adequate supply of blood to the body. The physiological state in which cardiac output is insufficient to meet the needs of the body or to do so only at a higher filing pressure. There are many underlying causes of HF, including myocardial infarction, coronary artery disease, valvular disease, hypertension, and myocarditis. Chronic heart failure is associated with neurohormonal activation and alterations in autonomic control. Although these compensatory neurohormonal mechanisms provide valuable support for the heart under normal physiological circumstances, they also play a fundamental role in the development and subsequent progression of HF.

For example, one of the body's main compensatory mechanisms for reduced blood flow in HF is to increase the amount of salt and water retained by the kidneys. Retaining salt and water, instead of excreting it via urine, increases the volume of blood in the bloodstream and helps to maintain blood pressure. However, the larger volumes of blood also cause the heart muscle, particularly the ventricles, to become enlarged. As the heart chambers become enlarged, the wall thickness decreases and the heart's contractions weaken, causing a downward spiral in cardiac function. Another compensatory mechanism is vasoconstriction of the arterial system, which raises the blood pressure to help maintain adequate perfusion, thus increasing the load that the heart must pump against.

In low ejection fraction (EF) heart failure, high pressures in the heart result from the body's attempt to maintain the high pressures needed for adequate peripheral perfusion. However, as the heart weakens because of such high pressures, the disorder becomes exacerbated. Pressure in the left atrium may exceed 25 mmHg, at which stage, fluids from the blood flowing through the pulmonary circulatory system transudate or flow out of the pulmonary capillaries into the pulmonary interstitial spaces and into the alveoli, causing lung congestion and if untreated the syndrome of acute pulmonary edema and death.

### Right Ventricle Failure

In one aspect, the present invention provides a method of treating right ventricle (RV) failure, comprising administering an integrin α5β1 inhibitor (e.g., small molecule compounds and antibodies disclosed herein). In some embodiments, the RV failure results from RV volume overload. In some embodiments, the RV volume overload is due to septal defects or valvular regurgitation. In some embodiments, the RV pressure overload is due to other WHO groups of pulmonary hypertension or outflow obstructions. In some embodiments, the RV failure is due to pulmonary artery stenosis.

In one aspect, the present invention provides a method of treating an RV cardiomyopathy comprising administering an integrin α5β1 inhibitor (e.g., small molecule compounds and antibodies disclosed herein). In some embodiments, the RV cardiomyopathy is due to infarction, arrythmia, or fibrosis.

In some embodiments, a5b1 inhibition (e.g., using an integrin α5β1 inhibitor). maintains cardiac output. In some embodiments, a5b1 inhibition prevents maladaptation of the RV. In some embodiments, administering the α5β1 inhibitor results in improved hypertrophy and/or fibrosis. In some embodiments, administering the α5β1 inhibitor prevents hypertrophy and/or fibrosis.

### Integrin Inhibitors

Integrins are a family of glycoprotein transmembrane receptors that mediate cell-cell and cell-matrix interactions. Integrins are heterodimers having two different chains, the alpha and beta subunits. In mammals, eighteen alpha and eight beta subunits have been described.

Integrin α5β1 is composed of subunits ITGA5 (integrin α5) and integrin β1. Several integrins bind to fibronectin. Integrin α5β1 is selective for fibronectin since it requires both the 9^{th} and 10^{th} type II repeats of fibronectin (FNIII-9 and FNIII-10) for interaction. Expression of α5β1 integrin is mainly in the vasculature and connective tissue. Expression is significantly enhanced in tumor blood vessels, but also in tumor cells itself of many types of cancer, including colon, breast, ovarian, lung and brain tumors. It is further expressed to varying degrees in many cell types including fibroblasts, hematopoictic cell, immune cells, smooth muscle cells, and epithelial cells. High expression of α5β1 integrin has also been observed fibrotic tissue such as pulmonary fibrosis.

In tissues, normal fibroblasts are present in low population of only 4-5%. However, during fibrosis they proliferate and can occupy up to 80-90% of the organ mass. Myofibroblasts in the fibrotic tissue produce large amounts of extracellular matrix proteins that make the tissue scarred and non-functional. Inhibition of myofibroblasts can counteract these processes. Integrins promote cell proliferation, survival, hypertrophic growth, and fibrosis. As described herein, integrin inhibition can modulate these key elements leading to the progression of pulmonary hypertension (e.g., PAH).

The present invention provides methods of treating a disease associated with increased expression or activity of integrin α5β1, comprising administering an integrin α5β1 inhibitor.

In one aspect, the present invention provides an integrin α5β1 inhibitor for use in treating a disease associated with increased expression or activity of integrin α5β1 in a subject in need of treatment thereof, comprising administering the integrin α5β1 inhibitor and a pharmaceutical excipient to the subject.

In some embodiments, the disease is characterized by the World Health Organization (WHO) group.

In some embodiments, the disease is pulmonary hypertension, WHO Group 1 pulmonary hypertension or pulmonary arterial hypertension (PAH), WHO Group 2pulmonary hypertension, WHO Group 3 pulmonary hypertension, WHO Group 4 pulmonary hypertension, and WHO Group 5 pulmonary hypertension.

In some embodiments, the disease is characterized by the World Health Organization (WHO) class system. In some embodiments, the disease is characterized by WHO functional class based on cardiac function. In some embodiments, the disease is pulmonary hypertension, WHO Class I pulmonary hypertension or pulmonary arterial hypertension (PAH), WHO Class II pulmonary hypertension, WHO Class III pulmonary hypertension, WHO Class IV pulmonary hypertension.

In some embodiments, the disease is heart failure or right ventricle failure.

In one aspect, the present invention provides a method of treating pulmonary arterial hypertension (PAH) in a subject, comprising administering an integrin α5β1 inhibitor.

### Integrin α5β1 Small Molecule Inhibitors

In some embodiments, the integrin α5β1 inhibitor is a small molecule compound that binds integrin α5β1.

In some embodiments, the integrin α5β1 inhibitor is a small molecule compound that specifically binds integrin α5.

In some embodiments, the integrin α5β1 inhibitor is a small molecule compound that specifically binds integrin β1.

In some embodiments, the integrin α5β1 inhibitor is a small molecule compound that specifically binds integrin α5β1.

In some embodiments, the integrin α5β1 inhibitor is a compound of Formula (I) or a pharmaceutically acceptable salt thereof,

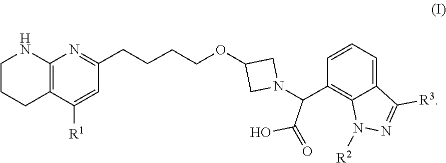

wherein:
R¹ is hydrogen or OMe;
R² is C₁₋₄alkyl optionally substituted with C₁₋₄alkoxy; and
R³ is C₁₋₄alkyl or C₃₋₅cycloalkyl.

In some embodiments, R2 is methyl or ethyl.

In some embodiments, R3 is methyl, ethyl, isopropyl, or cyclopropyl.

In some embodiments, R1 is hydrogen.

In some embodiments, the compound of Formula (I) is selected from:

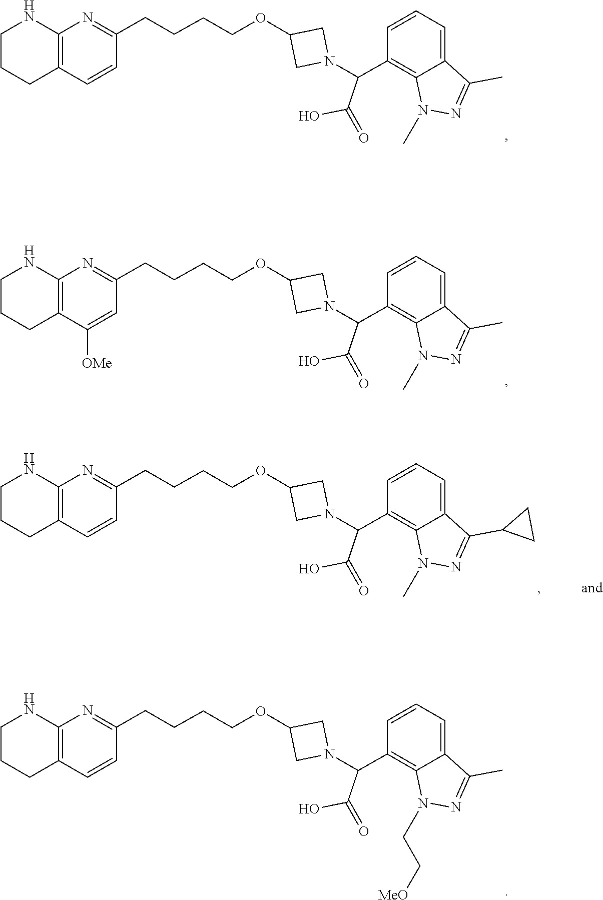 [SMILES: Cc1nn(C)c2c(C(C(=O)O)N3CC(OCCCCc4ccc5c(n4)NCCC5)C3)cccc12 ; COc1cc(CCCCOC2CN(C(C(=O)O)c3cccc4c(C)nn(C)c34)C2)nc2c1CCCN2 ; Cn1nc(C2CC2)c2cccc(C(C(=O)O)N3CC(OCCCCc4ccc5c(n4)NCCC5)C3)c21 ; COCCn1nc(C)c2cccc(C(C(=O)O)N3CC(OCCCCc4ccc5c(n4)NCCC5)C3)c21]

In some embodiments, the integrin α5β1 inhibitor is a compound is SMi:

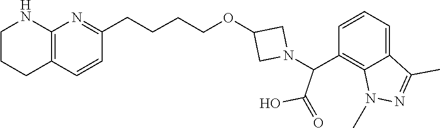 [SMILES: Cc1nn(C)c2c(C(C(=O)O)N3CC(OCCCCc4ccc5c(n4)NCCC5)C3)cccc12]

In some embodiments, the integrin α5β1 inhibitor is a dual inhibitor of α5β1 and αvβ1 (e.g., MRT).

### Integrin α5β1 Antibody Inhibitors

In some embodiments, the integrin α5β1 inhibitor is a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof.

In some embodiments, the integrin α5β1 inhibitor is an antibody.

In some embodiments, the integrin α5β1 inhibitor is an antibody that specifically binds integrin α5.

In some embodiments, the integrin α5β1 inhibitor is an antibody that specifically binds integrin β1.

In some embodiments, the integrin α5β1 inhibitor is an antibody that specifically binds integrin α5β1.

In some embodiments, the antibody is an integrin α5β1 antibody selected from the group consisting of volociximab (M200), PF-04605412 and MINT1526A.

In some embodiments, the antibody is the integrin α5β1 antibody volociximab (M200).

The antibody volociximab (M200) comprises a heavy chain amino acid sequence of SEQ ID NO: 1:

(SEQ ID NO: 1)
QVQLKESGPGLVAPSQSLSITCTISGFSLTDYGVHWVRQPPGKGLEWLV
VIWSDGSSTYNSALKSRMTIRKDNSKSQVFLIMNSLQTDDSAMYYCARH
GTYYGMTTTGDALDYWGQGTSVTVSSASTKGPSVFPLAPCSRSTSESTA
ALGCLVKDYFPEPVTVSWNSGALTSGVHTFPAVLQSSGLYSLSSVVTVP
SSSLGTKTYTCNVDHKPSNTKVDKRVESKYGPPCPSCPAPEFLGGPSVF
LFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHYT
QKSLSLSLGK

The antibody volociximab (M200) comprises a light chain amino acid sequence of SEQ ID NO: 2:

(SEQ ID NO: 2)
QIVLTQSPAIMSASLGERVTMTCTASSSVSSNYLHWYQQKPGSAPNLWI
YSTSNLASGVPARFSGSGSGTSYSLTISSMEAEDAATYYCHQYLRSPPT
FGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

In some embodiments, the antibody is the integrin α5β1 antibody M200. In some embodiments, the antibody is an integrin α5β1 antibody that competes for integrin binding with and/or binds the same epitope as M200.

In some embodiments, the antibody is the integrin α5β1 antibody PF-04605412. In some embodiments, the antibody is an integrin α5β1 antibody that competes for integrin binding with and/or binds the same epitope as PF-04605412.

In some embodiments, the antibody is an integrin α5β1 antibody described in WO2009100110A1, which is hereby incorporated by reference. In some embodiments, the antibody is the integrin α5β1 antibody competes for integrin binding and/or binds the same epitope as an integrin α5β1 antibody described in WO2009100110A1.

In some embodiments, the antibody is the integrin α5β1 antibody 22B5. In some embodiments, the antibody is an integrin α5β1 antibody that competes for integrin binding with and/or binds the same epitope as 22B5.

In some embodiments, the antibody is the integrin α5β1 antibody 24C7. In some embodiments, the antibody is an integrin α5β1 antibody that competes for integrin binding with and/or binds the same epitope as 24C7.

In some embodiments, the antibody is the integrin α5β1 antibody 1D9. In some embodiments, the antibody is an integrin α5β1 antibody that competes for integrin binding with and/or binds the same epitope as 1D9.

In some embodiments, the antibody is the integrin α5β1 antibody 2D2. In some embodiments, the antibody is an integrin α5β1 antibody that competes for integrin binding with and/or binds the same epitope as 2D2.

In some embodiments, the antibody is the integrin α5β1 antibody MINT1526A. In some embodiments, the antibody is the integrin α5β1 antibody 18C12 or an antibody derived from 18C12. In some embodiments, the antibody is the integrin α5β1 antibody h18C12.v6.1.5.

Exemplary integrin α5β1 antibodies are described in WO2010111254A1, which is hereby incorporated by reference. In some embodiments, the antibody is an integrin α5β1 antibody that competes for integrin binding and/or binds the same epitope as an integrin α5β1 antibody described in WO2010111254A1.

In some embodiments, the anti-α5β1 antibody comprises a VL domain comprising a CDR-L1 comprising TL-S/T-S/P/T-Q/N-H-F/S-T/I-Y-K/T-I-G/D/S; a CDR-L2 comprising L/I-N/T-S-D/H/S-G/S-S/L/T-H/Y-N/K/Q/I-K/T-G/A-D/S/V; a CDR-L3 comprising G/A-S/A/Y-S/Y-Y-S/A/Y-S/Y/T-GY-V/I, and a VH domain comprising a CDR-H1 comprising GFTFS-N/A-RW-I/V-Y; a CDR-H2 comprising GIKTKP-N/A/T-I/R-YAT-E/Q-YADSVKG and a CDR-H3 comprising L/V-TG-M/K-R/K-YFDY.

In some embodiments, the integrin α5β1 antibody competes for integrin binding and/or binds the same epitope as an antibody comprising a VL domain comprising a CDR-L1 comprising TL-S/T-S/P/T-Q/N-H-F/S-T/I-Y-K/T-I-G/D/S; a CDR-L2 comprising L/I-N/T-S-D/H/S-G/S-S/L/T-H/Y-N/K/Q/I-K/T-G/A-D/S/V; a CDR-L3 comprising G/A-S/A/Y-S/Y-Y-S/A/Y-S/Y/T-GY-V/I; and a VH domain comprising a CDR-H1 comprising GFTFS-N/A-RW-I/V-Y; a CDR-H2 comprising GIKTKP-N/A/T-I/R-YAT-E/Q-YADSVKG; and a CDR-H3 comprising L/V-TG-M/K-R/K-YFDY.

In some embodiments, the integrin α5β1 antibody comprises a VL domain comprising a CDR-L1 comprising TLSSQHSTYTI; a CDR-L2 comprising LNSDSSHNKGSGIPD; a CDR-L3 comprising AAYYAYGYV; and a VH domain comprises a CDR-H1 comprising GFTFSARWIY; a CDR-H2 comprising GIKTKPAIYATEYADSVKGRFT; and a CDR-H3 comprising LTGMKYFDY.

In some embodiments, the integrin α5β1 antibody competes for integrin binding with and/or binds the same epitope as an antibody comprising a VL domain comprising a CDR-L1 comprising TLSSQHSTYTI; a CDR-L2 comprising LNSDSSHNKGSGIPD; a CDR-L3 comprising AAYYAYGYV; and a VH domain comprises a CDR-H1 comprising GFTFSARWIY; a CDR-H2 comprising GIKTKPAIYATEYADSVKGRFT; and a CDR-H3 comprising LTGMKYFDY.

In some embodiments, the anti-α5β1 antibody comprises a VL domain comprising a CDR-L1 comprising TLSSQHSTYTIG a CDR-L2 LNSDSSHNKGS; a CDR-L3 comprising AAYYAYGYV; and a VH domain comprising a CDR-H1 comprising residues GFTFSARWIY; a CDR-H2 comprising residues GIKTKPAIYATEYADSVKG; and a CDR-H3 comprising residues LTGMKYFDY.

In some embodiments, the integrin α5β1 antibody competes for integrin binding and/or binds the same epitope as an anti-α5β1 antibody comprising a VL domain comprising a CDR-L1 comprising TLSSQHSTYTIG; a CDR-L2 LNSDSSHNKGS; a CDR-L3 comprising AAYYAYGYV; and a VH domain comprising a CDR-H1 comprising residues GFTFSARWIY; a CDR-H2 comprising residues GIKTKPAIYATEYADSVKG; and a CDR-H3 comprising residues LTGMKYFDY.

In some embodiments, the integrin α5β1 antibody is selected from an antibody described in Table 1. In some embodiments, the integrin α5β1 antibody competes for integrin binding and/or binds the same epitope as an anti-α5β1 antibody described in Table 1.

TABLE 1
Exemplary anti-α5β1 antibodies

| | LCDR1 | LCDR2 | LCDR3 | HDCR1 | HCDR2 | HCDR3 |
|---|---|---|---|---|---|---|
| 18C12 | TLSSQHST | LNSDGSH | GSSYSSG | GFTFSNR | GIKTKPNIYATEY | LTGMRYFDY |
| | YTIG | NKGD | YV | WIY | ADSVKG | |
| h18C12.v1.1 | TLSSQHST | LNSDGSH | GSSYSSG | GFTFSNR | GIKTKPNIYATEY | LTGMRYFDY |
| | YTIG | NKGD | YV | WIY | ADSVKG | |
| h18C12.v3 | TLSSQHST | LNSDGSH | GSSYSSG | GFTFSNR | GIKTKPNIYATEY | LTGMRYFDY |
| | YTIG | NKGD | YV | WIY | ADSVKG | |
| h18C12.v6 | TLSSQHST | LNSDGSH | AAYYAY | GFTFSNR | GIKTKPNIYATEY | LTGMKYFDY |
| | YTIG | NKGD | GYV | WIY | ADSVKG | |
| h18C12.v6.1. | TLSSQHST | LNSSGSHN | AAYYAY | GFTFSAR | GIKTKPAIYATEY | LTGMKYFDY |
| Lam3 | YTIG | KGS | GYV | WIY | ADSVKG | |
| h18C12.v6.2. | TLSSQHST | LNSSGSHN | AAYYAY | GFTFSAR | GIKTKPAIYATEY | LTGMKYFDY |
| Lam3 | YTIG | KGS | GYV | WIY | ADSVKG | |
| h18C12.v6.1. | TLSSQHST | LNSDSSHN | AAYYAY | GFTFSAR | GIKTKPAIYATEY | LTGMKYFDY |
| 5 | YTIG | KGS | GYV | WIY | ADSVKG | |
| h18C12.v7 | TLSSQHST | LNSDGSH | GYSYYS | GFTFSNR | GIKTKPNIYATEY | LTGMRYFDY |
| | YTIG | NKGD | GYV | WIY | ADSVKG | |
| h18C12.v9 | TLSSQHST | LNSDGSH | GSSYSTG | GFTFSNR | GIKTKPNIYATEY | LTGMRYFDY |
| | YTIG | NKGD | YV | WIY | ADSVKG | |
| h18C12.v15 | TLSPQHFT | LNSDGSH | GSSYSSG | GFTFSNR | GIKTKPNIYATEY | LTGMRYFDY |
| | YKID | NKGD | YV | WIY | ADSVKG | |
| h18C12.v16 | TLSSNHSI | LNSDGSH | GSSYSSG | GFTFSNR | GIKTKPNIYATEY | LTGMRYFDY |
| | YTIS | NKGD | YV | WIY | ADSVKG | |
| h18C12.v28 | TLSSQHST | LNSDGSH | GASYSS | GFTFSNR | GIKTKPNIYATEY | VTGMRYFDY |
| | YTIG | NKGD | GYI | WIY | ADSVKG | |
| h18C12.v30 | TLTTQHST | LNSDGSH | GSSYSSG | GFTFSNR | GIKTKPNIYATEY | LTGMRYFDY |
| | YTIG | NKGD | YV | WIY | ADSVKG | |
| h18C12.v51 | TLSSQHST | LNSDGSH | GSSYSSG | GFTFSNR | GIKTKPTRYATQY | LTGMRYFDY |
| | YTIG | NKGD | YV | WVY | ADSVKG | |
| h18C12.v54 | TLSSQHST | INSDGSHK | GSSYSSG | GFTFSNR | GIKTKPNIYATEY | LTGMRYFDY |
| | YTIG | KGV | YV | WIY | ADSVKG | |
| h18C12.v70 | TLSSQHST | LTSHGLH | GSSYSSG | GFTFSNR | GIKTKPNIYATEY | LTGMRYFDY |
| | YTIG | QKGV | YV | WIY | ADSVKG | |
| h18C12.v78 | TLSSQHST | LNSDSTYI | GSSYSSG | GFTFSNR | GIKTKPNIYATEY | LTGMRYFDY |
| | YTIG | TAV | YV | WIY | ADSVKG | |
| 22B5 | RASQSVSS | DASNRAT | QQRSNW | SSSYWG | SIYYSGRNYNNPS | HYYGSGSSYYY |
| | YLA | | PLT | | LKS | YDLDV |
| 24C7 | RASQSVS | DASNRAT | QQRTNW | SYAMH | VISFDGSNKNYAD | EYWGTYYYGM |
| | NYLA | | PYT | | SVKG | DV |
| 1D9 | RASQSVSS | DASNRAT | QQRSNW | STYAMH | VISYDGSNKYYAD | RESPPIYYYYG |
| | YLA | | PRT | | SVKG | MDV |
| 2D2 | RASQSVN | DASNRAT | QQRSNW | SYAMH | VISFDGSTKYYAD | EYWGTYYYGT |
| | SYLA | | PRT | | SVKG | DV |

In some embodiments, the antibody is an integrin α5β1 antibody 3C5 or 5B11. In some embodiments, the antibody is an integrin α5β1 antibody that competes for integrin binding with an antibody selected from 3C5 and 5B11. 3C5 and 5B11 are integrin aα5β1 antibodies described in WO2010072740A2, which is hereby incorporated by reference.

In some embodiments, the antibody is an integrin α5β1 antibody that competes for integrin binding with an antibody selected from the group consisting of volociximab (M200), P1D6, PF-04605412, MINT1526A, BMA5, BMB5, BMC5, HA5, JBS5, LS-C509074, LS-C24758, 1D9, 22B5, 24C7, 2D2, 3C2.2A8, 3C5, 5B11, MOR04055, MOR04624, P8D4, MOR04974, MOR04977, SG/19, and 18C12 (e.g., clone h18C12.v2.1 or h18C12.v6.1.5).

In some embodiments, the integrin inhibitor binds to α5β1. In some embodiments, the integrin inhibitor is a broad-spectrum integrin inhibitor. In some embodiments, the integrin inhibitor binds to α5β1 and one or more integrins.

In some embodiments, the α5β1 integrin inhibitor is selected from Table 2.

TABLE 2
Exemplary Integrin Inhibitors
Molecule

Non-Antibody Inhibitors
ATN-161
JSM6427
ATX-107
GLPG0187
SJ749
THR-687
Antibody Inhibitors
volociximab (M200)
PF-04605412
MINT1526A (RG-7594)

### Methods of Treatment

The present invention provides method of treating a heart or lung disease in a subject, comprising administering an integrin α5β1 inhibitor. In one aspect, the present invention provides a method of treating a disease associated with increased expression or activity of integrin α5β1, comprising administering an integrin α5β1 inhibitor. In some embodiments, the disease is characterized by the World Health Organization (WHO) group.

In some embodiments, the disease is pulmonary hypertension, WHO Group 1 pulmonary hypertension or pulmonary arterial hypertension (PAH), WHO Group 2 pulmonary hypertension, WHO Group 3 pulmonary hypertension, WHO Group 4 pulmonary hypertension, and WHO Group 5 pulmonary hypertension.

In some embodiments, the disease is characterized by the World Health Organization (WHO) class system. In some embodiments, the disease is characterized by WHO functional class based on cardiac function. In some embodiments, the disease is pulmonary hypertension, WHO Class I pulmonary hypertension or pulmonary arterial hypertension (PAH), WHO Class II pulmonary hypertension, WHO Class III pulmonary hypertension, WHO Class IV pulmonary hypertension.

In some embodiments, the disease is Persistent/recurrent Chronic Thromboembolic Pulmonary Hypertension (CTEPH) (WHO Group 4). In some embodiments, the disease is Pulmonary Arterial Hypertension (PAH) (WHO Group 1).

In some embodiments, the patient has Persistent/recurrent Chronic Thromboembolic Pulmonary Hypertension (CTEPH) (WHO Group 4) after surgical treatment or inoperable CTEPH. In some embodiments, patient has Pulmonary Arterial Hypertension (PAH) (WHO Group 1).

In some embodiments, the treatment is administered to improve exercise capacity and WHO functional class. In some embodiments, the treatment is administered to improve exercise capacity, improve WHO functional class and to delay clinical worsening.

In some embodiments, the disease is heart failure or right ventricle failure. In some embodiments, the disease is heart failure. In some embodiments, the disease is right ventricle failure.

In one aspect, the present invention provides a method of treating pulmonary arterial hypertension (PAH) in a subject, comprising administering an integrin α5β1 inhibitor.

In some embodiments, the integrin α5β1 inhibitor is administered orally, intravenously, subcutaneously, intranasally, transdermally, intraperitoneally, intramuscularly, or intrapulmonarily.

Also provided is a method for the treatment of a subject suffering from fibrosis or a fibrosis related disorder, comprising administering to said subject a therapeutically effective amount of integrin α5β1 inhibitor according to the invention. The term “fibrosis” as used herein refers to a condition characterized by a deposition of extracellular matrix components in the skin or organs, including lungs, kidneys, heart, liver, skin and joints, resulting in scar tissue. The term also refers to the process of formation of scar tissue.

In some embodiments, the fibrosis-related disorder is a disorder or condition which may occur as a result of fibrosis, or which is associated with fibrosis. In some embodiments, fibrosis and/or a fibrosis-related disorders is a disease or condition selected from the group consisting of kidney fibrosis, liver fibrosis, liver cirrhosis, pulmonary fibrosis, skin fibrosis, biliary fibrosis, peritoneal fibrosis, myocardial fibrosis, pancreatic fibrosis, bone marrow and/or myelofibrosis, reperfusion injury after hepatic or kidney transplantation, Interstitial Lung Disease (ILD), cystic fibrosis (CF), atherosclerosis, systemic sclerosis, osteosclerosis, spinal disc herniation and other spinal cord injuries, fibromatosis, fibromyalgia, arthritis, restenosis. Pulmonary fibrosis includes idiopathic pulmonary fibrosis and scleroderma lung fibrosis.

### Therapeutic Dose

In some embodiments, the method of treating a disease associated with increased expression or activity of integrin α5β1, comprises administering the integrin α5β1 inhibitor at a dose of 1 mg/kg to 1000 mg/kg. In some embodiments, the method of treating a heart or lung disease in a subject, comprises administering the integrin α5β1 inhibitor at a dose of 1 mg/kg to 1000 mg/kg. In some embodiments, embodiments, the disease is characterized by the World Health Organization (WHO) group as discussed above.

In some embodiments, the dose is at least 2 mg/kg, at least 4 mg/kg, at least 6 mg/kg, or at least 8 mg/kg. In some embodiments, the dose is at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg or at least 100 mg/kg. In some embodiments, the dose is at least 200 mg/kg, at least 300 mg/kg, at least 400 mg/kg, at least 500 mg/kg, at least 600 mg/kg, at least 700 mg/kg, at least 800 mg/kg, at least 900 mg/kg, or at least 1000 mg/kg.

### Cardiac Function Parameters

Various endpoint parameters can be assessed to determine efficacy of a treatment of the present invention, e.g., A5B1 level, pulmonary vascular resistance (PVR), mean pulmonary arterial pressure (PAP), cardiac index (CI), mean pulmonary capillary wedge pressure (PCWP), right atrial pressure (RAP), six-minute walk distance (6 MWD), brain natriuretic peptide (BNP) level, diffusion of lung capacity (DLCO), and death or survival. Sec, Chung et al. Chest (2010), 138(6):1383-1394.

PVR is commonly used as an endpoint parameter for determination of efficacy of treatment for PAH. A PVR of a subject of >240 dyn·sec/cm5 is an indication of mild PAH. A PVR of a subject of 600-800 dyn·sec/cm5 indicates moderate to severe PAH. After treatment using the methods and compositions of the invention, a decrease in PVR in a subject of 130 dyn·sec/cm5 or more indicates efficacious treatment. For example, administration of a A5B1 inhibitor to a subject with PAH that leads to a decrease in PVR of 180-350 dyn·sec/cm5 indicates efficacious treatment.

Mean pulmonary arterial pressure (PAP) is also used as an endpoint parameter to determine efficacy of treatment for PAH. A subject without PAH has a mean PAP ranging from about 15-24 mmHg. A subject having mild PAH has a mean PAP of about 25-30 mmHg (e.g., >25 mmHg at rest or 30 mmHg with exercise). A subject having severe PAH has a PAP of greater than 30 mmHg, for e.g., 40-70 mmHg or 60-70 mmHg. After treatment, a decrease in PAP of greater than 1.5 mmHg indicates efficacious treatment. In some embodiments, treatment leads to a decrease in PAP of greater than 5, 10, 20, 40, or 50 mmHg, which is indicative of efficacious treatment.

Cardiac index (CI) is also used as an endpoint parameter for determining efficacy of treatment for PAH. A low or decreased CI is indicative of heart failure. For e.g., a CI of 2.5 L/min/m2 or less is indicative of PAH or heart failure. After treatment, a CI increase of more than 0.3 L/min/m2 is indicative of efficacious treatment.

Mean pulmonary capillary wedge pressure (PCWP) can be used as an endpoint parameter for determining efficacy of treatment for PAH. A mean PCWP of less than or equal to 18 mmHg (e.g., less than or equal to 10 mmHg) indicates a subject having PAH. After treatment, an increase in mean PCWP of greater than 0.5 mmHg is indicative of efficacious treatment.

Right atrial pressure (RAP) is also used as an endpoint parameter to determine efficacy of treatment for PAH. A subject not suffering from PAH has a normal RAP of 0-8 mmHg. A RAP of 8 mmHg or greater is indicative of PAH. A subject suffering from severe PAH has a RAP of about 20 mmHg. After treatment, a decrease of greater than 0.5 mmHg is indicative of efficacious treatment.

Six-minute walk distance (6 MWD) is used as an endpoint parameter to determine efficacy of treatment of PAH. The mean 6 MWD of patients with CTD-PAH is about 300 m. After treatment, an increase in 6 MWD of 25 m or more, or greater than 10% increase indicates efficacious treatment. For example, after treatment, a 6 MWD of 1000 m or more indicates efficacious treatment.

Brain Natriuretic Peptide (BNP) is used as an endpoint parameter to determine efficacy of treatment of PAH. BNP is a sensitive marker for the worsening of heart failure and is a predictor of mortality in PAH patients. Normal levels of BNP are <100 pg/mL, e.g., 30-90 pg/mL. Higher levels of BNP indicate worsening of heart failure. A BNP level of about 100-200 pg/mL, e.g., 160 pg/mL or higher, indicates early heart failure. A BNP level of about 200-1000 pg/mL indicates real heart failure. The mean BNP level of CTD-PAH patients is about 430 pg/mL. After treatment, any reduction in BNP level indicates efficacious treatment.

N-Terminal pro Brain Natriuretic Peptide (NT-proBNP): Reproducible, noninvasive parameters are useful in following patients with PAH. BNP is produced in the cardiac ventricles and is elevated in PPH/IPAH. BNP levels have recently been shown to be closely related to functional impairment in PPH/IPAH patients and parallel the extent of pulmonary hemodynamic changes and right heart failure. BNP levels longitudinally correlate with the functional assessments being made over the course of the study. Plasma NT-pro-BNP are measured by a sandwich immunoassay using polyclonal antibodies that recognize epitopes located in the N-terminal segment (1 to 76) of pro-BNP (1 to 108) (Elecsys analyzer, Roche Diagnostics, Manheim, Germany).

Diffusion of lung capacity (DLCO), or diffusion capacity of CO, is also used as an endpoint parameter to determine efficacy of treatment of PAH. DLCO measures the ability of carbon monoxide (CO) to diffuse across membranes. A subject not suffering from PAH has a normal DLCO of greater than 80%. A subject suffering from PAH has an abnormal DLC of less than 80%, less than 65%, or less than 45%. After treatment, any increase in % DLCO indicates efficacious treatment.

In some embodiments, administration of the integrin α5β1 inhibitor modulates the level of a biomarker in the subject, wherein the biomarker is selected from the group consisting of survivin, PCNA, Ki67, and annexin V.

### SoC and Combination Therapy

As described herein, a method of treating a disease associated with increased expression or activity of integrin α5β1, comprising administering an integrin α5β1 inhibitor can include a combination therapy in which a patient in need of treatment is administered an integrin a5b1 inhibitor in combination with one or more drugs approved for the treatment of PH, PAH, heart failure, or right ventricle failure.

Approved drugs currently used in the treatment of PH, PAH, heart failure and right ventricle failure in the US or the European Union (EU) include the orally administered PDE-5 inhibitors: sildenafil (Revatio) and tadalafil (Adeirca); the dual endothelin-1A receptor antagonist (ERA): bosentan (Tracleer), ambrisentan (Letairis in US; Volibris internationally). Patients with more advanced disease are often treated with prostacyclins or prostacyclin analogs such as iloprost (Ventavis) or treprostinil (Tyvaso) given as multiple daily inhalations, epoprostenol (Flolan/Veletri) or treprostinil (Remodulin) given as continuous intravenous infusions, or treprostinil also used as a continuous subcutaneous infusion. Intravenous injection of sildenafil is approved for patients who are currently prescribed but are temporarily unable to take oral sildenafil. Inhaled nitric oxide (INOmax) is approved for the neonatal form of PAH—persistent pulmonary hypertension of the newborn (PPHN). Thus, in accordance with the invention, combination therapies of any of these drugs and an integrin a5b1 inhibitor are useful in the treatment of PAH or a disorder disclosed herein.

In some embodiments, the second therapy is selected from the group consisting of anticoagulants, diuretics, a digitalis glycosideglycosides, calcium channel blockers, endothelin receptor antagonists, phosphodiesterase 5 (PDE5) inhibitors, prostanoids, prostanoids receptor agonists, soluble guanylate cyclase stimulators, and/or surgery.

In some embodiments, the second therapy is oxygen, Warfarin, furosemide, bumetanide, bendroflumethiazide, metolazone, spironolactone, amiloride, Digoxin, nifedipine, diltiazem, nicardipine, amlodipine, ambrisentan, bosentan, macitentan, sildenafil, tadalafil, epoprostenol, iloprost, treprostinil, riociguat, selexipag, surgery, pulmonary endarterectomy, and/or atrial septostomy.

In some embodiments, the second therapy is macitentan and/or tadalafil.

Flolan (prostacyclin analog) is an approved therapy for PAH but is extremely cumbersome and inconvenient to use (intravenous), and has unique safety concerns. As a result, Flolan is usually reserved for patients with severe functional status or rapidly progressive PAH. Patients must constitute the drug in sterile conditions several times daily. The drug is available as a freeze-dried preparation that needs to be dissolved in alkaline buffer. Because of its short half-life (3-5 min) and stability (8 h at room temperature), Flolan must be maintained in a refrigerated state while given by continuous infusion through a central venous catheter via a portable pump that is worn in a bag around the waist (CADD pump, Smith's Medical MD, St. Paul, Minn.). In 2008, the FDA also approved a new continuous intravenous formulation of epoprostenol that is stable at room temperature for up to 24 h after dilution and may be stored up to 5 days at refrigerator temperature before use (GeneraMedix Inc., Liberty Corner, N.J.). In 2009, GeneraMedix Inc. sold this formulation to Actelion, which began to market the drug (under the brand name Veletri) in April 2010. In late 2010, the Veletri label was expanded to allow preparation of medication up to 7 days at refrigerator temperature or up to 48 h at room temperature in advance of use. Thus, in one embodiment of the invention, an integrin a5b1 inhibitor is administered in combination with epoprostenol, in any of its approved forms, to treat PAH.

Remodulin (continuous subcutaneous infusion form of prostacyclin analog) was not generally used as initial therapy because of its expense, route of delivery, and limited efficacy. In 2004, the FDA and Health Canada approved an intravenous formulation of Remodulin for patients with PAH class II-IV disease who cannot tolerate the subcutaneous form. In early 2006, the FDA expanded the Remodulin label to include patients requiring transition from Flolan. In 2009, United Therapeutics received FDA approval for an inhaled formulation of treprostinil (Tyvaso). Thus, in one embodiment of the invention, an integrin a5b1 inhibitor is administered in combination with treprostinil to treat PAH.

Ventavis (iloprost), a prostacyclin analogue administered via inhalation is also marketed in several member countries of the EU as Ilomedine as an intravenous formulation. The label for inhaled iloprost in the EU is restricted to patients with idiopathic PAH and functional class III symptoms. In contrast, the label in the US is broader: patients with PAH (regardless of etiology) and class III or IV symptoms. It is required 6 to 9 times a day administration. Thus, in one embodiment of the invention, an integrin a5b1 inhibitor is administered in combination with iloprost, in any of its approved forms, to treat PAH.

In 2001, the nonselective ERA Tracleer (bosentan) became the first oral PAH therapy and was available only through a special centralized access program in the US because of its significant risk of (reversible) liver injury, teratogenicity, testicular atrophy, and male sterility. Treatment with Tracleer consists of an initial dosage of 62.5 mg twice daily for 4 weeks, followed by a maintenance dose of 125 mg twice daily. Tracleer was initially indicated for patients with PAH and moderate or severe functional status (WHO class III, IV). In 2008 (EU) and 2009 (US), the label was expanded to patients with mild symptoms (functional class II). Thus, in one embodiment of the invention, an integrin a5b1 inhibitor is administered in combination with bosentan, in any of its approved forms, to treat PAH.

Ambrisentan is the oral selective ERA-receptor antagonist marketed by Gilead Sciences in the US (Letairis) and by GlaxoSmithKline in other regions (Volibris) for the once-daily treatment of patients with WHO class II or III symptoms to improve exercise capacity and delay clinical worsening. As with bosentan, ambrisentan has class effects of teratogenicity, testicular injury, reduced male fertility, and anemia. Thus, in one embodiment of the invention, an integrin a5b1 inhibitor is administered in combination with ambrisentan, in any of its approved forms, to treat PAH.

The oral PDE-5 inhibitor Revatio (sildenafil) was approved in the US for the treatment of PAH (WHO Group I) to improve exercise ability and delay of clinical worsening at a dose of 20 mg three times daily, regardless of functional class or etiology. The EU label is restricted to improvement of exercise capacity in patients with PAH, which is either idiopathic or associated with collagen vascular disease and with functional class III status. In 2009, the FDA approved an intravenous form of Revatio given as an injection (10 mg 3-times a day) for a patient unable to take the oral formulation. In May 2010, the EU approved Revatio as an oral suspension (compounded from 20 mg tablets) for the treatment of pediatric patient aged 1 to 17 years with PAH. Thus, in one embodiment of the invention, an integrin a5b1 inhibitor is administered in combination with sildenafil, in any of its approved forms, to treat PAH.

The oral PDE-5 Inhibitor Adeirca (tadalafil) 40 mg once daily is indicated in the US to improve exercise ability in patients with PAH (WHO Group I) regardless of etiology or functional class (Packet Insert). The EU label is restricted to patients with functional class II and III status. Tadalafil has a long half-life (35 h) in patients with PAH (US Packet Insert) has also shown benefit in patients with PAH on concomitant bosentan.

Thus, the method of treating the patient may involve administering at least one additional active agent, i.e., in addition to an integrin a5b1 inhibitor. The additional active agent may be, for example, a vasodilator such as prostacyclin, epoprostenol, and sildenafil; an endothelin receptor antagonist such as bosentan; a calcium channel blocker such as amlodipine, diltiazem, and nifedipine; an anticoagulant such as warfarin; a diuretic, a prostanoid (e.g., prostacyclin or PGI2), drugs for treating diseases associated with overactive B cells or dysfunctional B cells such as Rituximab, and/or a Type V phosphodiesterase (PDE5) inhibitor.

When the method of the invention involves combination therapy, i.e., wherein a secondary agent such as a vasodilator is co-administered with an integrin a5b1 inhibitor, the agents may be administered separately, at the same, or at different times of the day, or they may be administered in a single composition. Thus, the present invention provides novel pharmaceutical formulations in which an integrin a5b1 inhibitor is combined with one of the active agents discussed above and unit dose forms of those formulations.

In the combination therapies of the invention, each agent can be administered in an “immediate release” manner or in a “controlled release manner.” When the additional active agent is a vasodilator, for instance, any dosage form containing both active agents i.e., both the integrin a5b1 inhibitor and the vasodilator, can provide for immediate release or controlled release of the vasodilator, and either immediate release or controlled release of an integrin a5b1 inhibitor.

As a general example, a combination dosage form of the invention for once-daily administration might contain in the range of about 1 mg to about 1000 mg of an integrin a5b1 inhibitor of an integrin a5b1 inhibitor, in a controlled release (e.g., sustained release) or immediate release form, and either sildenafil in immediate release form, or in controlled release form, with the additional active agent present in an amount that provides a weight ratio of an integrin a5b1 inhibitor to sildenafil, or a weight ratio of an integrin a5b1 inhibitor to sildenafil, specified as above. In other formulations of the invention, two or more additional active agents, which may or may not be in the same class of drug (e.g., vasodilators), can be present in combination, along with an integrin a5b1 inhibitor. In such a case, the effective amount of either or each individual additional active agent present will generally be reduced relative to the amount that would be required if only a single added agent were used.

The additional active agent may also be, as discussed above, a Type V phosphodiesterase inhibitor, administered with an integrin a5b1 inhibitor, or with both the integrin a5b1 inhibitor and a vasodilator. Examples of Type V phosphodiesterase inhibitors include, without limitation, avanafil, sildenafil, tadalafil, zaprinast, dipyridamole, vardenafil and acid addition or other pharmaceutically acceptable salts thereof. Sildenafil is an excellent example. In an exemplary embodiment, an integrin a5b1 inhibitor is co-administered with a Type V phosphodiesterase inhibitor selected from the group consisting of avanafil, tadalafil, and sildenafil, and the daily dose of a compound of the integrin a5b1 inhibitor is a given above for the monotherapeutic regimen.

In one embodiment, the vasodilator is selected from sildenafil, avanafil, tadalafil, zaprinast, dipyridamole, vardenafil, bosentan, and pharmaceutically acceptable salts thereof.

The additional active agent may also be, as discussed above, an endothelin receptor antagonist, e.g., bosentan, sitaxsentan, or ambrisentan, with bosentan being an exemplary active agent.

A pharmaceutical composition of the invention is a pharmaceutical formulation containing an active agent formulated in a manner compatible with its intended route of administration. A variety of routes are contemplated, including but not limited to, oral, pulmonary, inhalational, sublingual, intranasal, parenteral, intradermal, transdermal, topical, transmucosal, subcutaneous, intravenous, intramuscular, intraperitoneal, buccal, rectal, and the like. The term “parenteral” as used herein is intended to include subcutaneous, intravenous, and intramuscular injection.

Generally, pharmaceutical formulations of the invention are prepared for oral administration and in an immediate release form suitable for once per day (QD) administration. Certain formulations are suitable for intranasal administration to a patient.

Certain pharmaceutical formulations of the invention comprise an integrin a5b1 inhibitor or a salt thereof and one or more pharmaceutically acceptable (approved by a state or federal regulatory agency for use in humans, or is listed in the U.S. Pharmacopia, the European Pharmacopia) excipients or carriers. The term excipient or carrier as used herein broadly refers to a biologically inactive substance used in combination with the active agents of the formulation. An excipient can be used, for example, as a solubilizing agent, a stabilizing agent, a diluent, an inert carrier, a preservative, a binder, a disintegrant a coating agent, a flavoring agent, or a coloring agent. Preferably, at least one excipient is chosen to provide one or more beneficial physical properties to the formulation, such as increased stability and/or solubility of the active agent(s). An integrin a5b1 inhibitor or a salt thereof as described herein is an exemplary active agent suitable for use in the formulations of the present invention.

Examples of suitable excipients include certain inert proteins such as albumins; hydrophilic polymers such as polyvinylpyrrolidone; amino acids such as aspartic acid (which may alternatively be referred to as aspartate), glutamic acid (which may alternatively be referred to as glutamate), lysine, arginine glycine, and histidine; fatty acids and phospholipids such as alkyl sulfonates and caprylate; surfactants such as sodium dodecyl sulphate and polysorbate; nonionic surfactants such as such as TWEEN®, PLURONICS®, or polyethylene glycol (PEG); carbohydrates such as glucose, sucrose, mannose, maltose, trehalose, and dextrins, including cyclodextrins; polyols such as mannitol and sorbitol; chelating agents such as EDTA; and salt-forming counter-ions such as sodium.

Solutions or suspensions used for the delivery can include the following components: a sterile diluent such as water for injection, saline solution, fixed oils, polyethylene glycols, glycerine, propylene glycol, polysorbate, tocopherol polyethylene glycol succinate (TPGS), or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. These preparations can be enclosed in ampoules, disposable syringes or multiple dose vials made of glass or plastic.

In some embodiments, the pharmaceutical formulations of the present invention contain a plurality of liposomes or microparticles comprising the integrin a5b1 inhibitor active agent. In various embodiments, the pharmaceutical formulation of the integrin a5b1 inhibitor is a powder comprising solid particles (e.g., liposomes or microparticles) suitable for administration via inhalation. The solid particles comprise the active agent, a carrier, optionally a surfactant, and optionally additional recipients. The powder may be prepared by any convenient method. An example of a preparatory method is spray drying a solution containing the active agent (and other components) onto a powder comprising the carrier compound. Another example is freeze drying a solution comprising all of the components of the final powder.

Suitable liposomes for use in the present formulations of the invention are known in the art. For example, suitable liposomes include cholesterol, 1,2-distearoyl-sn-glycero-3-phosphocholine (DSPC) and PEG-DSPE, with the weight ratio being about 5:10:1. In some embodiments, the liposome formulation comprises about 0.1-25%, e.g., 0.1%, 1%, 5%, 10% or 20% (w/w) of a phospholipid, such as dipalmitoylphosphatidylcholine (DPPC) and 1,2-distearoyl-sn-glycero-3-phosphocholine (DSPC). In some embodiments, the liposome formulation comprises about 0.5-20%, e.g., 1%, 5%, or 10% (w/w) of a hydrophilic polymer, such as polyvinylpyrrolidone (PVP). In some embodiments, the liposome formulation comprises about 10-35% of an amino acid, such L-leucine.

Suitable microparticles for use in the formulations of the invention are known in the art. For example, microparticles are formed of one or more hydrophilic polymers such as polyvinylpyrrolidone (e.g., PVP-10), polyvinyl alcohol (e.g., PVA-30), polyvinyl acetate, or Poloxamer (e.g., Poloxamer-188). In some embodiments, the microparticle formulation comprises about 70-85 wt % of polyvinyl alcohol (e.g., PVA-30), about 5-15% PVP (e.g., PVP-10), 1-5% Poloxamer (e.g., Poloxamer-188), 0-10% L-leucine, and about 0.5-10% of an integrin a5b1 inhibitor compound (e.g., 5%). In some embodiments, the formulation is suitable for administration via the respiratory tract.

The pharmaceutical formulations of an integrin a5b1 inhibitor useful in the methods of the invention can be prepared as a liquid or in a solid form such as a powder, tablet, pill, or capsule for oral administration. Liquid formulations of the invention may take such forms as suspensions, solutions, or emulsions in oily or aqueous vehicles, and may contain formulatory agents such as suspending, stabilizing and/or dispersing agents. In one embodiment, the formulation is an aqueous solution. In another embodiment, the final formulation is lyophilized. In some embodiments, integrin a5b1 inhibitor is formulated for inhalation.

In various embodiments, the formulations of the invention comprise an integrin a5b1 inhibitor at a concentration of from 0.25 wt % to 100 wt %, or from 0.25 wt % in 50 wt %, or from 0.8 wt % to 25 wt %, or from 1 wt % to 10%, or from 1.5 wt % to 5 wt %. In certain embodiments, an integrin a5b1 inhibitor compound is formulated at a concentration of from about 0.5 wt % to about 5 wt %. In certain embodiments, an integrin a5b1 inhibitor compound is formulated at a concentration of about 0.25 wt % to about 10 wt %.

The present invention also provides a pharmaceutical pack or kit comprising one or more containers filled with a solid or liquid formulation of an integrin a5b1 inhibitor. In a particular embodiment, the formulation is a powder formulation of an integrin a5b1 inhibitor. In various embodiments, an integrin a5b1 inhibitor is formulated at a concentration of at least about 0.5 wt % and the formulation is suitable for delivery via inhalation to a human.

The present invention also provides for a use of a formulation of an integrin a5b1 inhibitor in the manufacture of a medicament for treating PAH, or a disorder disclosed herein, in a subject in need thereof. Generally, the pharmaceutical formulation is sterile.

Generally, the dosage forms, e.g., an inhalable dosage form, provide for sustained release, i.e., gradual, release of a compound of the current invention, for e.g., an integrin a5b1 inhibitor, from the dosage form to the patient's body over an extended time period, typically providing for a substantially constant blood level of the agent over a time period in the range of about 4 to about 12 hours, typically in the range of about 6 to about 10 hours. In a particularly preferred embodiment, there is a very gradual increase in blood level of the drug following nasal administration of the dosage form containing a compound of the current invention, for e.g., an integrin a5b1 inhibitor, such that peak blood level is not reached until at least 4-6 hours have elapsed, with the rate of increase of blood level drug approximately linear. In addition, in the preferred embodiment, there is an equally gradual decrease in blood level at the end of the sustained release period.

Although the pharmaceutical compositions of the invention are preferably formulated for inhalation, e.g., as a solution in saline, as a dry powder, or as an aerosol, other modes of administration are suitable as well. For example, administration may be sublingual, oral, parenteral, transdermal, via an implanted depot, transmucosal, e.g., rectal or vaginal, preferably using a suppository that contains, in addition to the active agent, excipients such as a suppository wax. Transmucosal administration also encompasses transurethral administration, as described, for example, in U.S. Pat. Nos. 5,242,391; 5,474,535 and 5,773,020 to Place et al.

Depending on the intended mode of administration, the pharmaceutical formulation may be a solid, semi-solid or liquid, such as, for example, a tablet, as capsule, a caplet, a liquid, a suspension, an emulsion, a suppository, granules, pellets, beads, a powder, or the like, preferably in unit dosage form suitable for single administration of a precise dosage. Suitable pharmaceutical compositions and dosage forms may be prepared using conventional methods known to those in the field of pharmaceutical formulation and described in the pertinent texts and literature, e.g., in Remington: The Science and Practice of Pharmacy (Easton, Pa.: Mack Publishing Co., 1995). For those compounds that are orally active, oral dosage forms are generally preferred, and include tablets, capsules, caplets, solutions, suspensions and syrups, and may also comprise a plurality of granules, beads, powders, or pellets that may or may not be encapsulated. Preferred oral dosage forms are tablets and capsules.

In embodiments, it may be especially advantageous to formulate compositions of the invention in unit dosage form for case of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of an integrin a5b1 inhibitor, such that the unit dosage in each tablet or capsule is approximately 50% of therapeutically effective amount.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like.

Capsules are another oral dosage forms for those compounds of the current invention, for e.g., an integrin a5b1 inhibitors, that are orally active, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited earlier herein, which describes materials and methods for preparing encapsulated pharmaceuticals.

Oral dosage forms, whether tablets, capsules, caplets, or particulates, if desired, may be formulated so as to provide for controlled release of the compounds of the current invention, for e.g., an integrin a5b1 inhibitors, and in a preferred embodiment, the present formulations are controlled release oral dosage forms.

Generally, as will be appreciated by those of ordinary skill in the art, sustained release dosage forms are formulated by dispersing the active agent within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Preparations according to this invention for parenteral administration include sterile aqueous and nonaqueous solutions, suspensions, and emulsions. Injectable aqueous solutions contain the active agent in water-soluble form. Examples of nonaqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

The active agent may also be administered through the skin using conventional transdermal drug delivery systems, wherein the active agent is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer.

In addition to the formulations described previously, the active agent may be formulated as a depot preparation for controlled release of the active agent, preferably sustained release over an extended time period. These sustained release dosage forms are generally administered by implantation (e.g., subcutaneously or intramuscularly or by intramuscular injection).

Certain compounds or active agents of the present invention are capable of further forming salts. All of these forms are also contemplated within the scope of the claimed invention.

The compounds of the present invention can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

Certain compounds of the present invention can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a subject. Prodrugs in the present invention are prepared by modifying functional groups present in the compound in such a way that the modifications are cleaved, either in routine manipulation or in vivo, to the parent compound. Prodrugs include compounds of the present invention wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the invention, and the like, See Bundegaard, H., Design of Prodrugs, p 1-92, Elesevier, New York-Oxford (1985).

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

In some embodiments, the composition is suitable for inhalation. In one embodiment, the composition is an inhalable formulation used for treating PAH, or a disorder disclosed herein.

In still another aspect, the present disclosure provides a pharmaceutical composition comprising an integrin a5b1 inhibitor and a plurality of particles, wherein the plurality of particles is a plurality of liposomes comprising 1,2-distearoyl-sn-glycero-3-phosphoethanolamine-N-[amino(polyethylene glycol)] (PEG-DSPE) or a plurality of microparticles comprising a hydrophilic polymer. In one embodiment, the composition is suitable for inhalation, in one embodiment, the composition is an inhalable formulation used for treating PAH, or a disorder disclosed herein.

### Compositions

The pharmaceutical compositions described herein can be administered in a variety of different ways. Examples include administering a pharmaceutical composition comprising a peptide according to the invention or multimeric, preferably peptide according to the invention and containing a pharmaceutically acceptable carrier via oral, intranasal, rectal, topical, intraperitoneal, intravenous, intramuscular, subcutaneous, subdermal, transdermal, intrathecal, and intracranial methods. For oral administration, the active ingredient can be administered in solid dosage forms, such as capsules, tablets, and powders, or in liquid dosage forms, such as elixirs, syrups, and suspensions.

Pharmaceutical compositions according to the invention comprise at least one pharmaceutically acceptable carrier, diluent or excipient. Examples of suitable carriers for instance comprise keyhole limpet haemocyanin (KLH), serum albumin (e.g., BSA or RSA) and ovalbumin. In some embodiments said suitable carrier is a solution, for example saline. Examples of excipients which can be incorporated in tablets, capsules and the like are the following: a binder such as gum tragacanth, acacia, corn starch or gelatine; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. When the dosage unit form is a capsule, it may contain, in addition to materials of the above type, a liquid carrier such as fatty oil. Various other materials may be present as coatings or to otherwise modify the physical form of the dosage unit. For instance, tablets may be coated with shellac, sugar or both. A syrup or elixir may contain the active compound, sucrose as a sweetening agent, methyl and propyl parabens as preservatives, a dye and a flavoring such as cherry or orange flavor. A pharmaceutical composition according to the invention is preferably suitable for human use.

Sterile compositions for injection can be formulated according to conventional pharmaceutical practice by dissolving or suspending the integrin a5b1 inhibitor of the invention in a vehicle for injection, such as water or a naturally occurring vegetable oil like sesame oil, coconut oil, peanut oil, cottonseed oil, etc., or a synthetic fatty vehicle like ethyl oleate or the like. Buffers, preservatives, antioxidants and the like may also be incorporated.

Compositions for topical administration can also be formulated according to conventional pharmaceutical practice. “Topical administration” as used herein refers to application to a body surface such as the skin or mucous membranes to locally treat conditions resulting from microbial or parasitic infections. Examples of formulations suitable for topical administration include, but are not limited to a cream, gel, ointment, lotion, foam, suspension, spray, aerosol, powder aerosol. Topical medicaments can be epicutaneous, meaning that they are applied directly to the skin. Topical medicaments can also be inhalational, for instance for application to the mucosal epithelium of the respiratory tract, or applied to the surface of tissues other than the skin, such as eye drops applied to the conjunctiva, or ear drops placed in the car. Said pharmaceutical composition formulated for topical administration preferably comprises at least one pharmaceutical excipients suitable for topical application, such as an emulsifier, a diluent, a humectant, a preservatives, a pH adjuster and/or water.

### EXAMPLES

The following examples describe some of the preferred modes of making and practicing the present invention. However, it should be understood that these examples are for illustrative purposes only and are not meant to limit the scope of the invention.

### Example 1: Integrin α5β1 Inhibitors Impair Proliferation in hPASMCs on Fibronectin

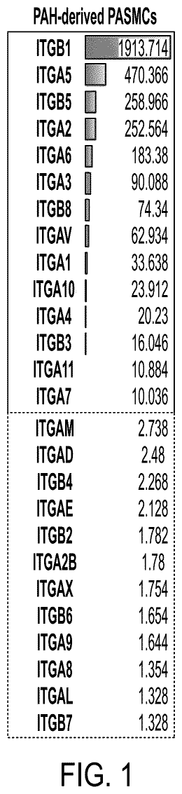

This example demonstrates in vitro effects of integrin α5β1 inhibitors on hPASMCs in vitro. RNA expression of human integrins expressed in PAH-patient derived pulmonary arterial smooth muscle cells was determined using NanoString detect integrin targets (FIG. 1).

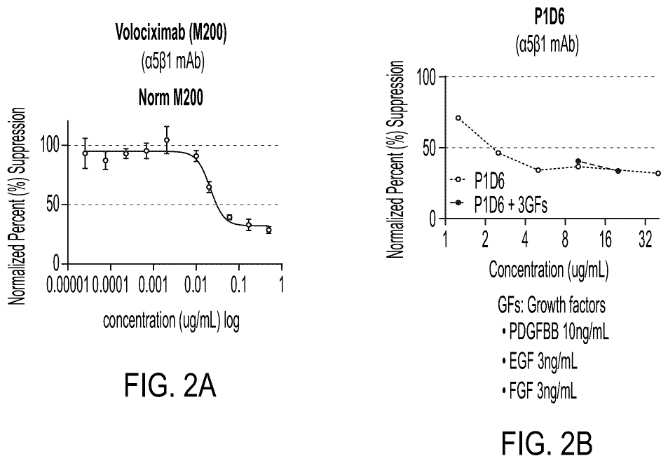

Antibody integrin α5β1 inhibitors were evaluated to determine whether integrin α5β1 inhibitors (e.g., P1D6 antibody or M200 antibody) could impair proliferation. Cells were seeded at 3000 cells/well in a 96 well plate coated with fibronectin. The next day integrin α5β1 inhibitor treatments were added and proliferation was monitored for up to 5 days. Integrin expressing hPASMCs treated with anti-integrin a5b1 antibodies, M200 (FIG. 2A) and P1D6 (FIG. 2B) demonstrated reduced proliferation. Selective inhibition of a5b1 resulted in approximately 70% reduction of hPASMCs proliferation.

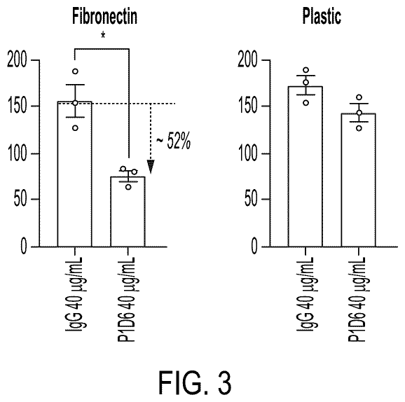

As shown in FIG. 3 proliferation of hPASMCs treated with P1D6 was only reduced fibronectin-coated plates suggesting that inhibition of proliferation blocking a5b1 is fibronectin-dependent.

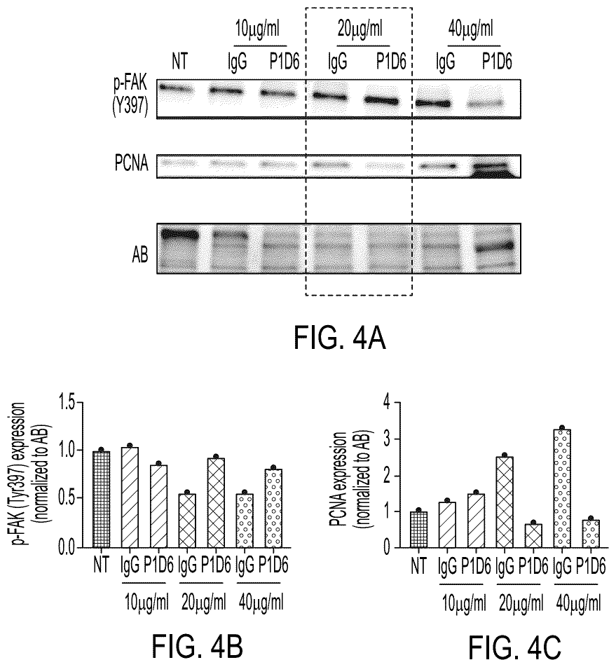

Dose response experiments with P1D6 were performed to determine the range of cellular specificity of P1D6. PAH-PASMCs were treated with P1D6 at doses of 10 μg/ml, 20 μg/ml and 40 μg/ml on fibronectin coated plates. Media was changed every 48 hours and assessed for efficacy markers at 48, 72, and 120 hours. FIG. 4A-4C shows exemplary western blot analysis measuring expression levels of p-FAK (FIG. 4B) and PCNA (FIG. 4C) in PAH-PASMCs at 72 hours following exposure to P1D6 on fibronectin coated plates.

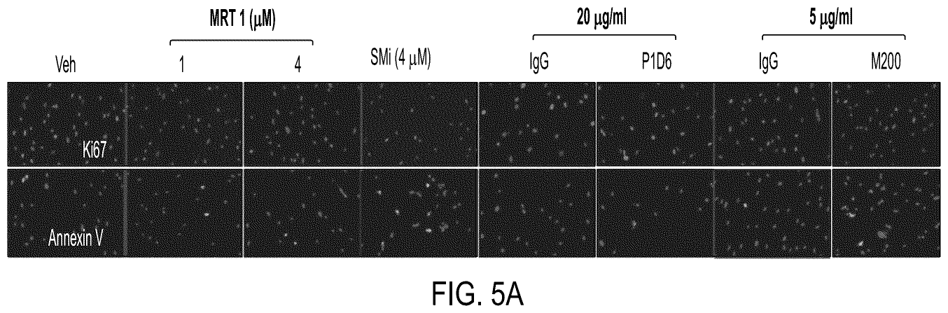

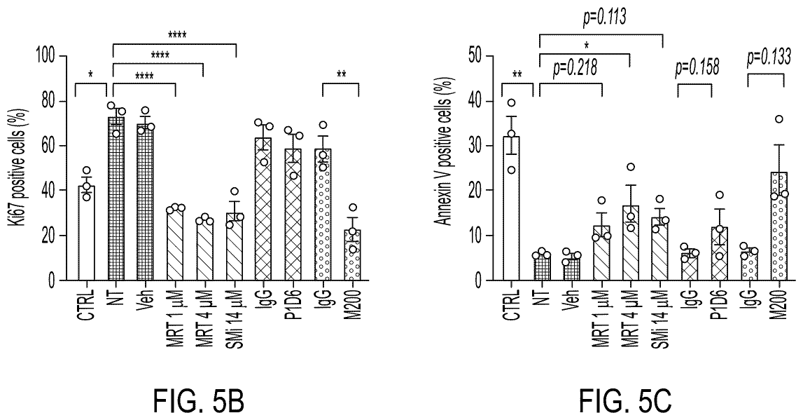

Integrin α5β1 inhibitors (MRT, SMi, P1D6, or M200) were assessed for proliferation and apoptosis on Fb-coated plates. FIG. 5A-5C show exemplary proliferation (Ki67) and apoptosis (annexin V) of PAH-PASMCs treated with integrin α5β1 inhibitors (MRT, SMi, P1D6, or M200).

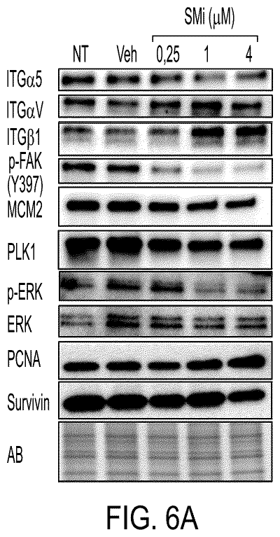

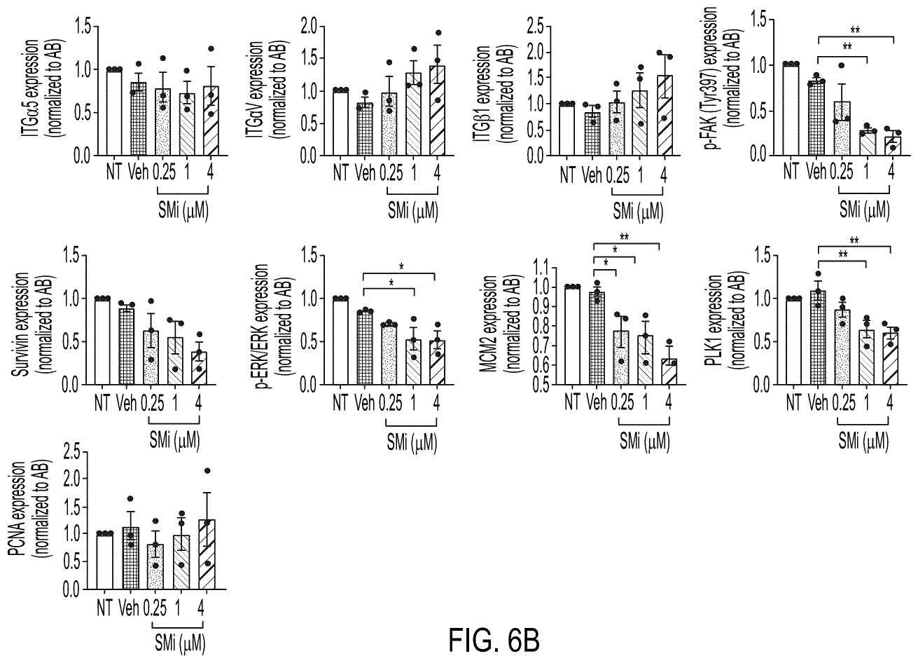

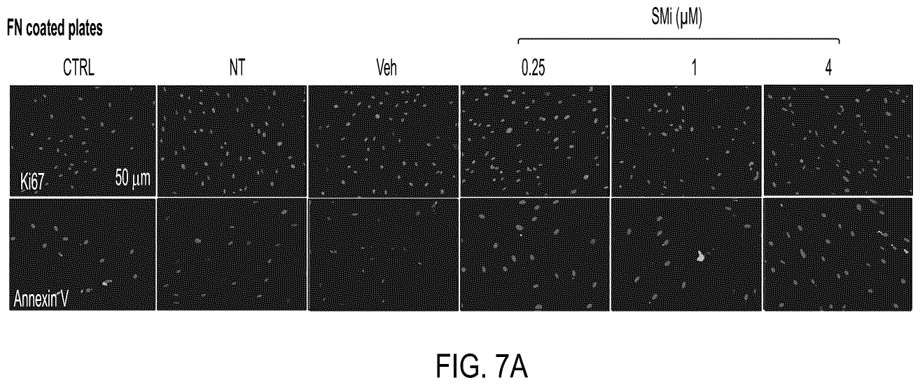

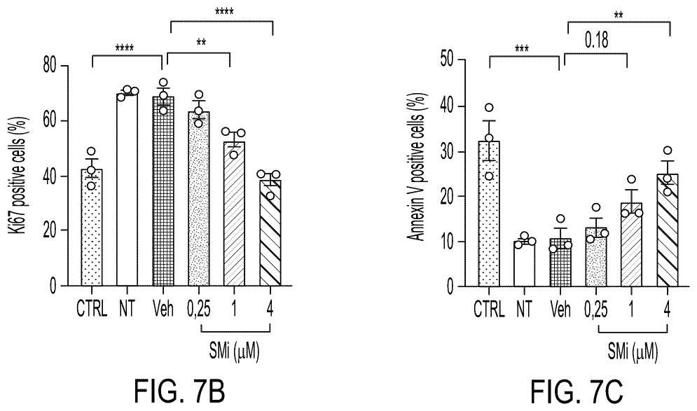

PAH-PASMCs were treated with integrin α5β1 inhibitor SMi at 0.25 μM, 1 μM, and 4 μM on Fb-coated plates. Cells were evaluated by Western blot analysis to determine expression levels of ITGα5, ITGαV, ITGβ1, p-FAK, MCM2, PLK1, p-ERK, ERK, PCNA and Survivin in hPASMCs (FIG. 6A-6B). As shown in FIG. 7A-7C, SMi treatment impaired proliferation and increased apoptosis in a dose-dependent manner.

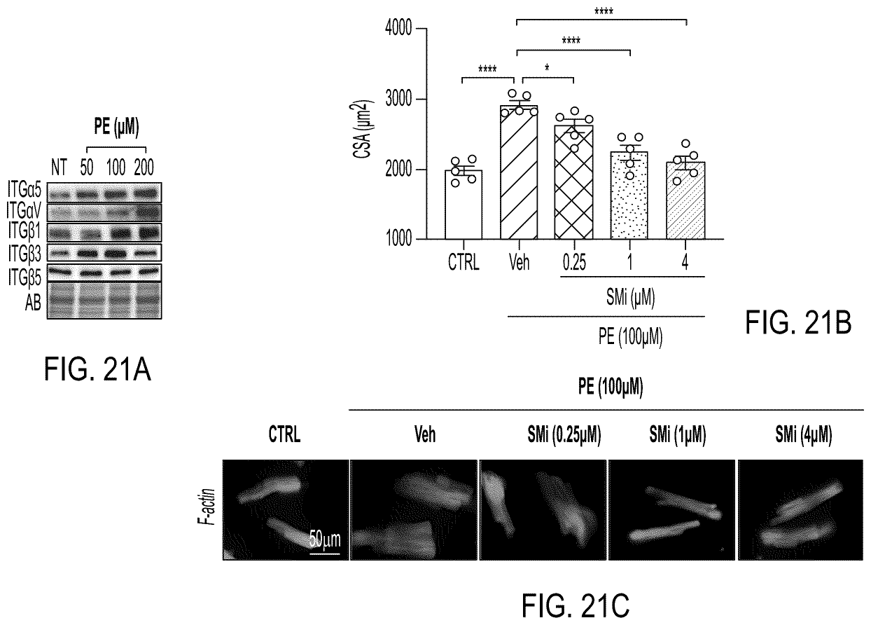

Primary rat cardiomyocytes were treated with 50 μM, 100 μM, or 200 μM, phenylephrine (PE). Expression of ITGα5, ITGαV, ITGβ1, ITGβ3, ITGβ5 were determined by western blot (FIG. 21A). Cells treated with 100 μM PE were simultaneously exposed to integrin α5β1 inhibitor SMi at 0.25 μM, 1 μM, and 4 μM. As shown in FIG. 21B and 21C, inhibition of α5β1 prevents PE-induced adult rat cardiomyocytes hypertrophy.

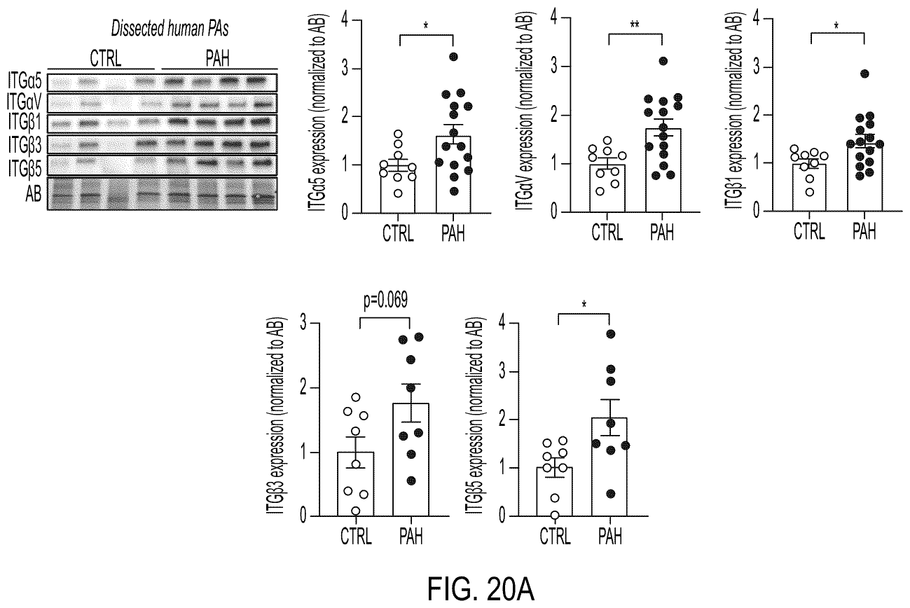

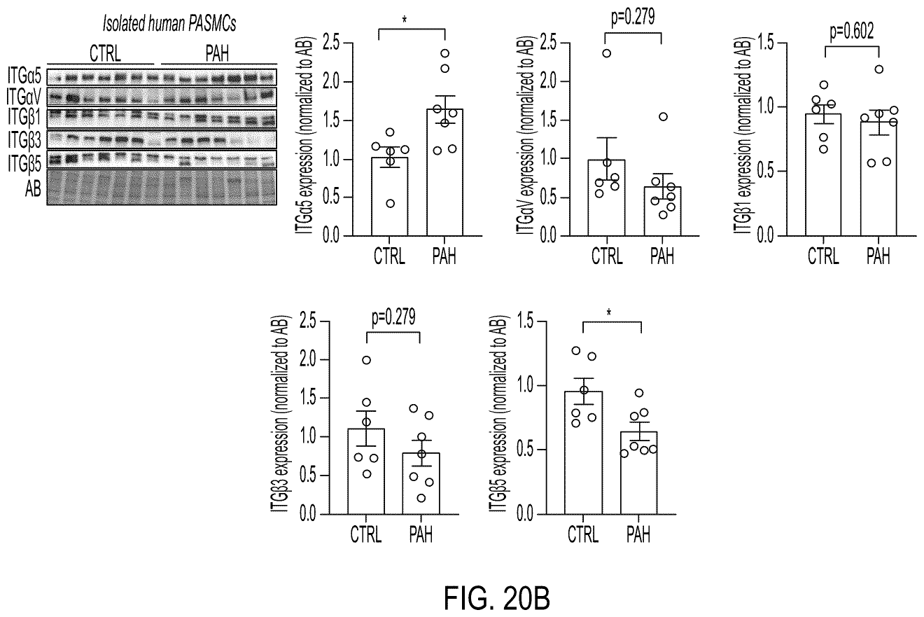

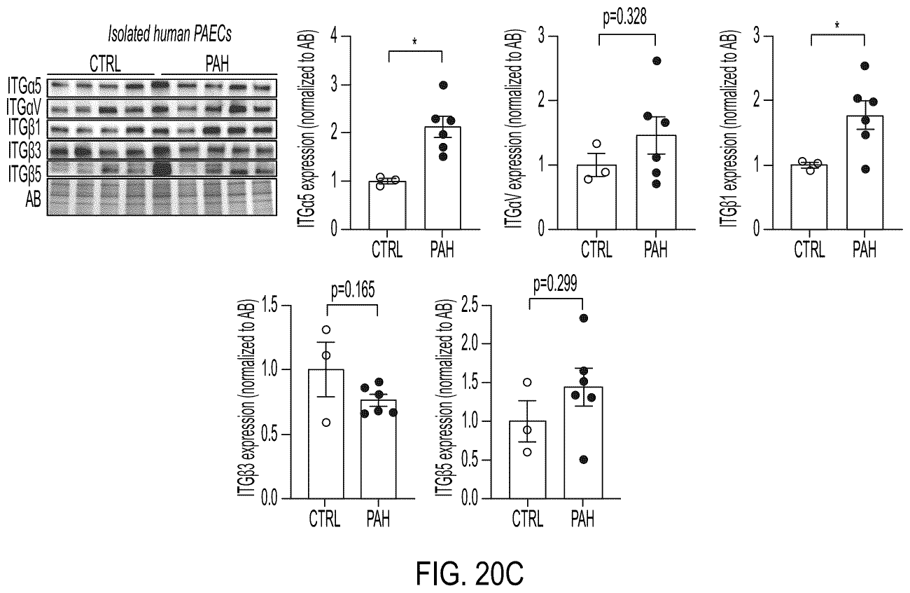

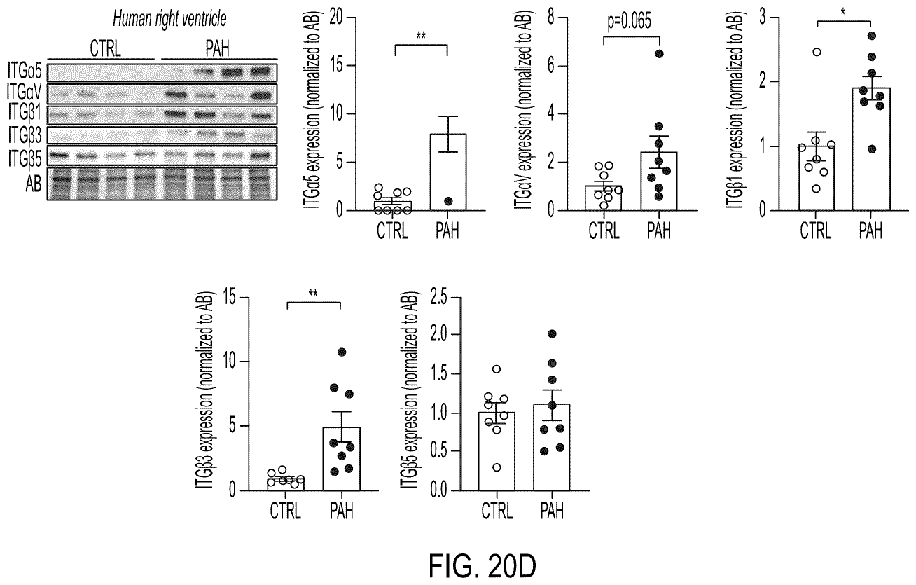

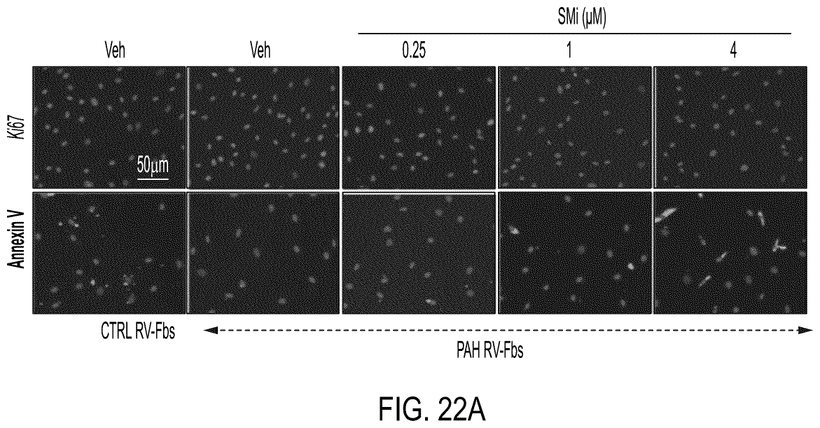

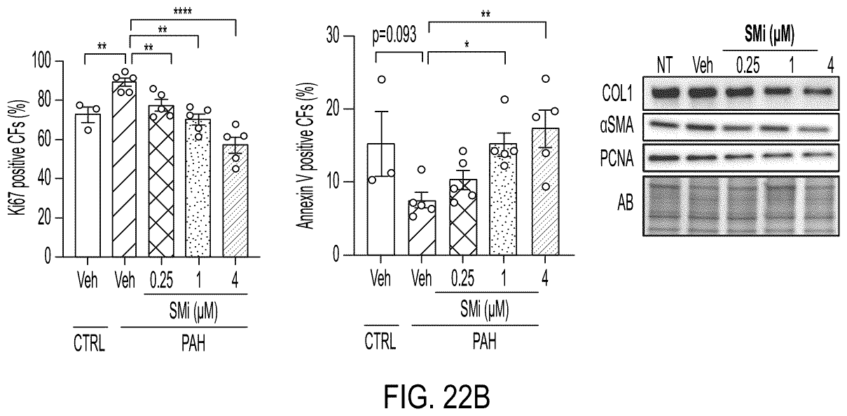

PAH patient samples were assessed for expression of fibronectin binding integrins. As shown in FIG. 20A-20D, expression of certain integrins is significantly changed in dissected PAs, isolated PASMCs, isolated PAECs and decompensated RV from PAH patients compared to controls. Human PAH-RVFbs were treated with integrin α5β1 inhibitor SMi at 0.25 μM, 1 μM, and 4 μM. Pharmacological inhibition with SMi demonstrated that integrin α5β1 decreases PAH-RVFbs proliferation and activation in a dose dependent manner. (FIGS. 22A and 22B).

### Example 2: In Vivo Efficacy of Integrin α5β1 Inhibitors in Sugen-hypoxia Animal Model

This example demonstrates efficacy of integrin α5β1 inhibitors in Sugen/Hypoxia (SuHx) induced PAH rat model alone and in combination with Standard of care (SoC) therapies. Broad-spectrum pharmacological inhibition of α5β1 improves hemodynamics and vascular remodeling in Su/Hx rats with established PAH alone or in combination with standard of care.

Animals were injected with 20 mg/kg Sugen 5416 at day 0 and subjected to hypoxia conditions (10% oxygen) for 3 weeks. Echocardiography was performed at the start of treatment with integrin α5β1 inhibitors at week 3 and at week 5. Right heart catheterization was performed at the end of week 5. Tissues were harvested for analysis.

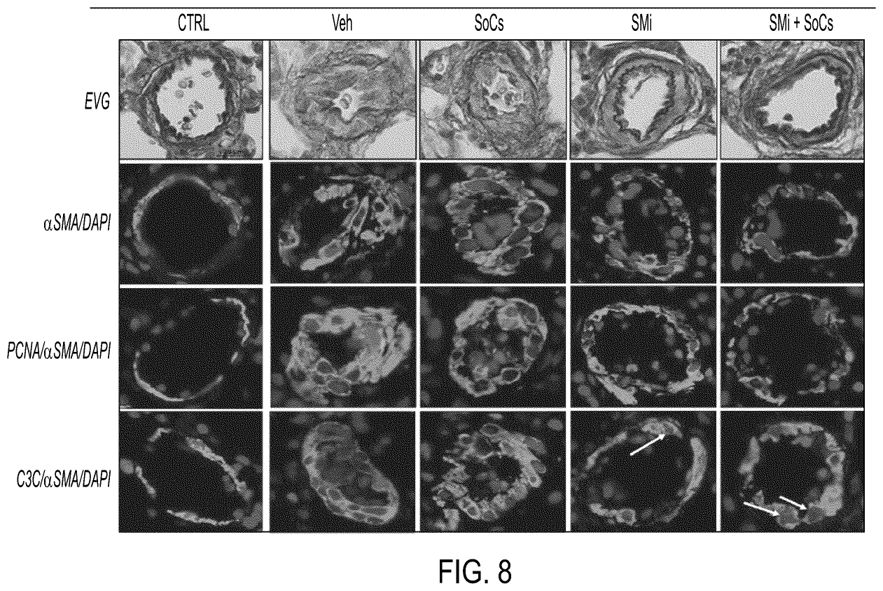

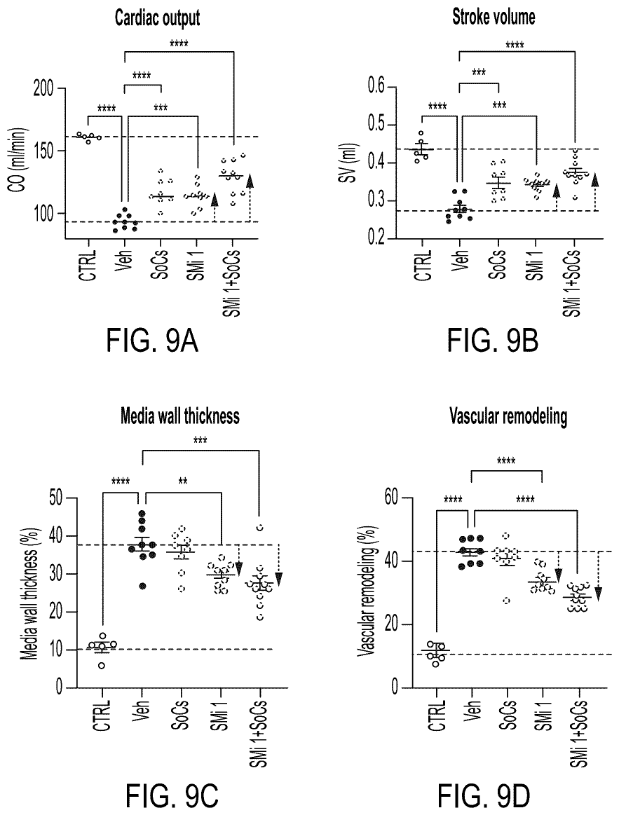

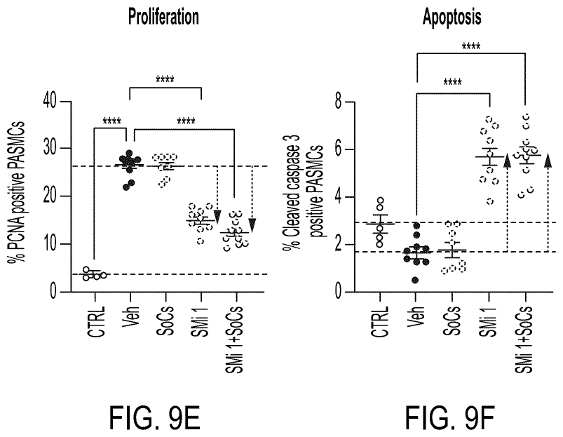

As shown in FIG. 8 by exemplary histology staining of EVG, αSMA, PCNA, and C3C, SMi demonstrated vascular remodeling in Sugen/Hypoxia (SuHx)-induced PAH rats. Treatment with SMi and/or vasodilator SoCs (e.g., Macitentan (Maci) and Tadalafil (Tada)) improved cardiac output (FIG. 9A), stroke volume (FIG. 9B), media cell wall thickness (FIG. 9C), vascular remodeling (FIG. 9D) proliferation (FIG. 9E) and apoptosis (FIG. 9F) were measured. Dotted lines indicate the disease window.

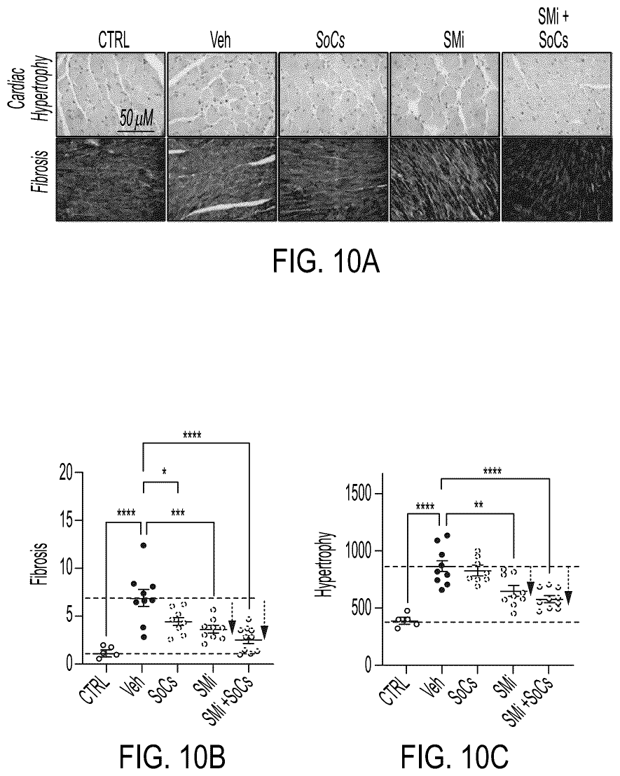

As shown in FIG. 10A-10C, treatment with SMi and/or SoCs (e.g., Macitentan (Maci) and Tadalafil (Tada)) reduced hypertrophy and fibrosis in SuHx rats.

### Integrin α5β1 Inhibition Improves Cardiac Function in SuHx Animals

To determine the selectivity of SMi for α5β1, SuHx animals were prepared as described above and treated with dual α5β1/αvβ1 inhibitor (MRT). Animals were divided into the following groups:
1) Non-SuHx vehicle PO BID (n=5)
2) SuHx+vehicle PO BID (PBS) (n=10)
3) SuHx+MRT 60 mpk PO BID (n=10)
4) SuHx+Macitentan 1 mg/Kg +Tadalafil 10 mg/Kg daily (n=10)
5) SuHx+MRT 60 mpk PO BID +Macitentan 1 mg/Kg +Tadalafil 10 mg/Kg daily (n=10)

MRT treated SuHx rats are assessed for improved cardiac function vascular remodeling demonstrated by EVG staining, media cell wall thickness, mean pulmonary arterial pressure, cardiac output, and right ventricular fractional area change.

### Integrin α5β1 Antibody Inhibitors in Sugen/hypoxia (SuHx) Mice Model of Pulmonary Arterial Hypertension (PAH)

SuHx treated mice were prepared as described above and treated with integrin α5β1 antibody inhibitors (e.g., anti-mouse integrin α5β1 clone 339.1) or SMi. anti-mouse integrin α5β1 clone 339.1 is described in Bhaskar, V., Zhang, D., Fox, M. et al. A function blocking anti-mouse integrin α5β1 antibody inhibits angiogenesis and impedes tumor growth in vivo. J Transl Med 5, 61 (2007). doi.org/10.1186/1479-5876-5-61), which is hereby incorporated by reference in its entirety.

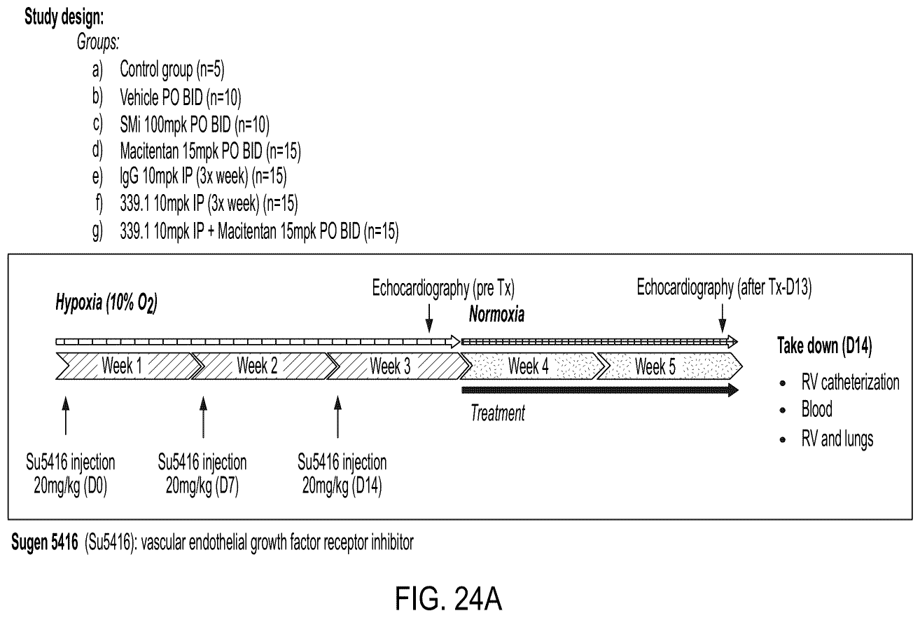

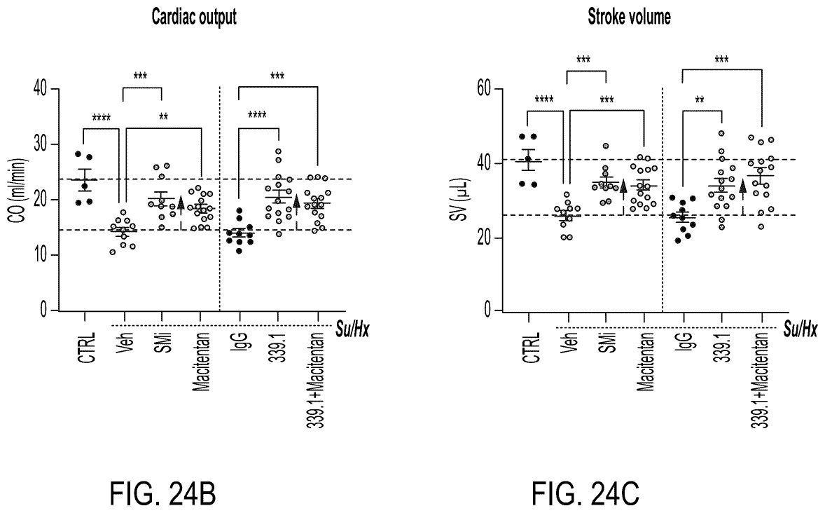

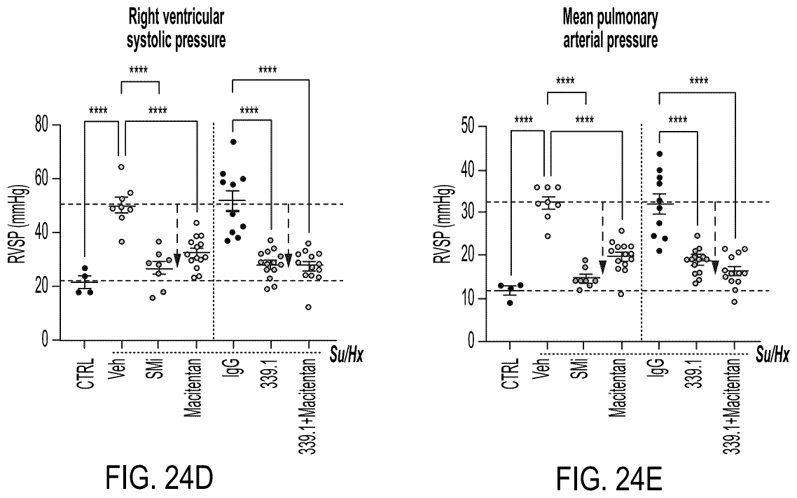

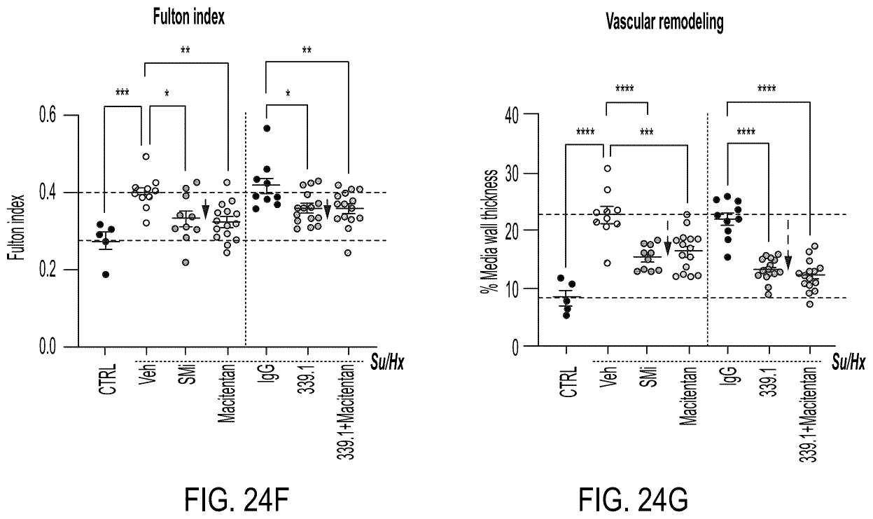

Mice were injected with 20 mg/kg Sugen 5416 (vascular endothelial growth factor receptor inhibitor) at day 0, day 7 and day 14 and subjected to hypoxia conditions (10% oxygen) for 3 weeks. Echocardiography was performed at the start of treatment with integrin α5β1 inhibitors at week 3 (e.g., day 21) and at the end of week 5. Right heart catheterization was performed at the end of week 5. Tissues were harvested for analysis as summarized in FIG. 24A.

Anti-Integrin α5β1 antibody treated SuHx rats were assessed for improved cardiac function vascular remodeling demonstrated by EVG staining, media cell wall thickness, mean pulmonary arterial pressure, cardiac output and right ventricular fractional area change. Cardiac function was measured including improved cardiac output, and stroke volume. Other features including media cell wall thickness, vascular remodeling, proliferation, and apoptosis were measured.

Mice were divided into the following treatment and control groups:
Control group (n=5)
Vehicle PO BID (n=10)
SMi 100 mpk PO BID (n=10)
Macitentan 15 mpk PO BID (n=15)
IgG isotype control10 mpk IP (3× week) (n=15)
339.1 10 mpk IP (3× week) (n=15)
339.1 10 mpk IP+Macitentan 15 mpk PO BID (n=15)

As shown in FIGS. 24B-24G, anti-α5β1 mouse antibody (antibody 339.1) phenocopied the cardiac and vascular improvement of SMi. These results confirm that α5β1 plays a key role in PAH and that integrin α5β1 inhibitors can be used to treat PAH.

Robust effects of the a5b1 inhibitors in the right ventricles of SuHx animals demonstrated by a reduction of RVSP and TAPSE, the amelioration of cardiomyocyte hypertrophy, and a decrease in cardiac fibrosis. Treatment with a5b1 inhibitors led to a significantly increased cardiac output, suggesting a functional improvement of the right heart.

### Example 3: In Vivo Efficacy of Integrin α5β1 Inhibitors in MCT Studies

This example demonstrates efficacy of compound SMi treatment in monocrotaline (MCT) rat model.

MCT Animals were divided into study groups as follows:
(1) Non-MCT vehicle orally twice a day (BID) (n=6)
(2) MCT+PBS vehicle orally twice a day (BID) (n=8)
(3) MCT+SMi at 100 mpk orally BID (n=8)

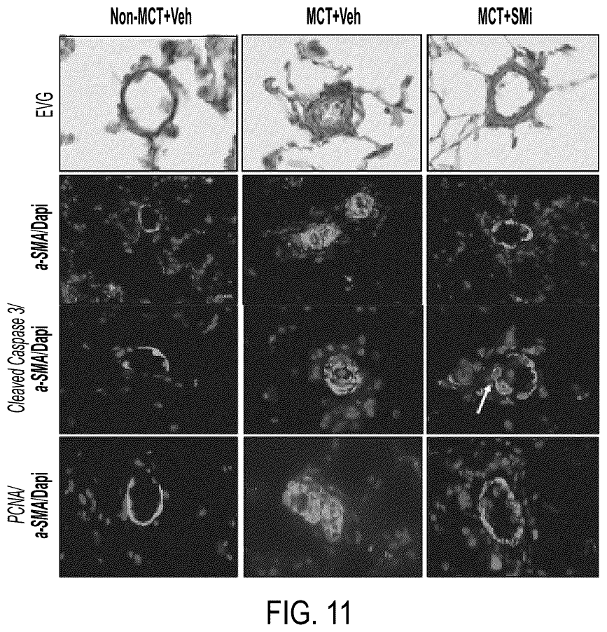

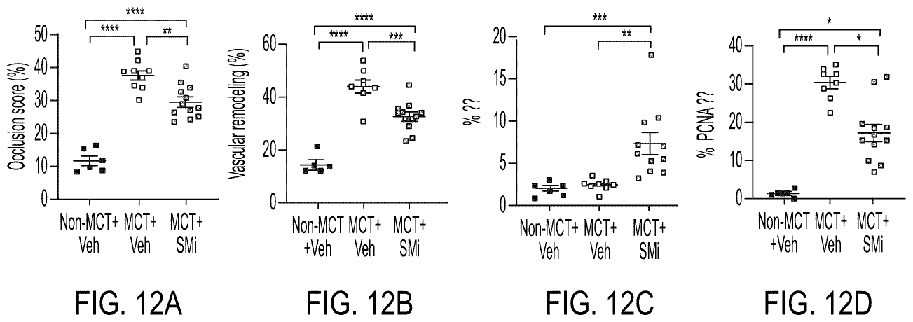

MCT (60 mg/kg) was administered by subcutaneous injection. Animals were exposed to vehicle or SMi treatment for 2 weeks. Blood, RV and lung analysis was performed. Cells were assessed for expression of EVG, αSMA, PCNA, and C3C. As shown in FIG. 11, animals treated with SMi showed improved vascular remodeling in monocrotaline (MCT) rat pulmonary hypertension model. FIG. 12A-12D show exemplary results of treatment with SMi in MCT rats. Occlusion score (FIG. 12A), vascular remodeling (FIG. 12B), apoptosis (FIG. 12C) and proliferation (FIG. 12D) were measured.

To test the impact of SMi on cardiac function, MCT animals with a cardiac defect were treated with SMi or sildenafil as follows (1) Non-MCT vehicle PO BID; (2) MCT+vehicle PO BID (3) MCT+SMi 100 mg/Kg PO BID (4) MCT+sildenafil 10 mg/Kg PO BID (5) MCT+sildenafil 30 mg/Kg PO BID (6) MCT+sildenafil 100 mg/Kg PO BID. Pulmonary hypertension phenotype was not induced in this study (MCT causes both vascular and cardiac injury). Treatment with SMi, vehicle or sildenafil was administered, twice a day, from Day 14 to Day 27. Echocardiogram monitoring of the progression of the disease was carried out on Day 0, Day 14 and on surgery day (Day 28) for all the animals. Blood samples were taken on Day 0, Day 14 and Day 28.

Pulmonary artery maximum velocity, cardiac output, stroke volume, right ventricle anterior wall thickness and pulmonary artery acceleration time were determined. An echocardiograph (Model Vivid E9 with XDclear Ultrasound, GE Healthcare, Illinois, United States) connected to a 13.0 MHz linear transducer was used to measure the pulmonary artery maximum velocity (Vmax), the artery pulmonary velocity time integral (VTI), the pulmonary artery diameter, the heart rate, the right ventricle anterior wall thickness and the pulmonary artery acceleration time. The data were used to calculate the cardiac output and the stroke volume as follows:

The cardiac output was calculated using the following formula (JF Lewis et al. 1984):

Cardiac output=heart rate×VTI((pulmonary artery diameter²×3.1416)/4)

The stroke volume was calculated using the following formula:

Stroke volume=cardiac output/heart rate

A considerable increase in cardiac output by SMi in this MCT model was observed suggesting a dissociation of cardiac benefits from the effects on pulmonary resistance. Treatment with SMi resulted in a direct improvement in the heart by a5b1 inhibition that is independent of its reverse remodeling effects in pulmonary arterioles.

### Combination Therapy of Integrin α5β1 Inhibitors and SoC in MCT Animals

MCT animals were treated with SMi alone or in combination with SoC therapy (Macitentan (1 mg/kg)+Tadalafil (10 mg/kg) daily). MCT Animals were divided into study groups as follows:
(1) Non-MCT vehicle orally twice a day (BID) (n=6)
(2) MCT+PBS vehicle orally twice a day (BID) (n=8)
(3) MCT+SMi at 100 mpk orally BID (n=8)
(4) MCT+SMi at 100 mpk orally BID+SoC (Macitentan (1 mg/kg)+Tadalafil (10 mg/kg) daily (n=8).

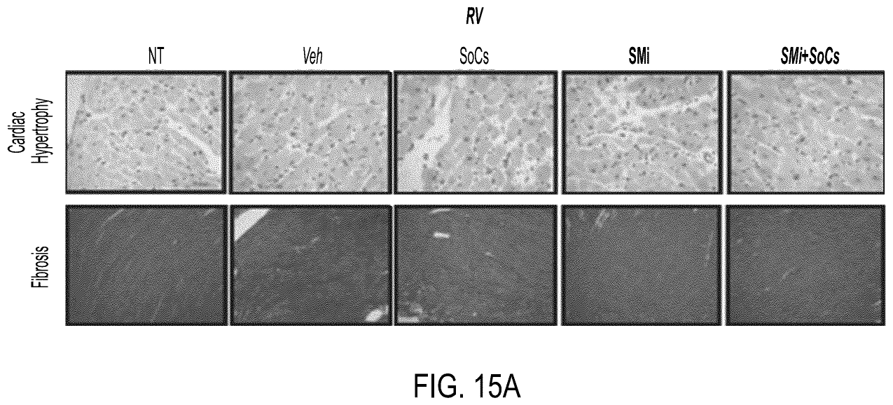

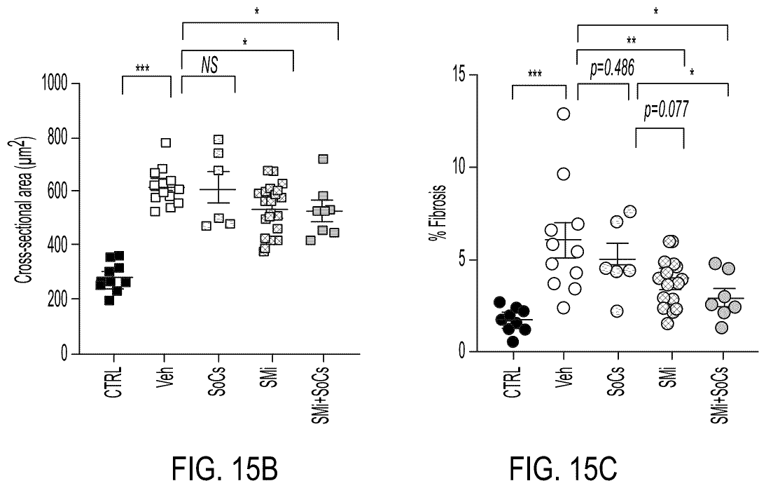

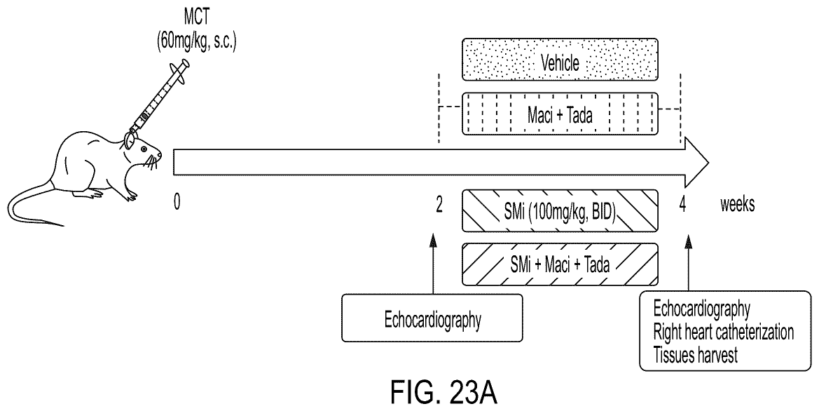

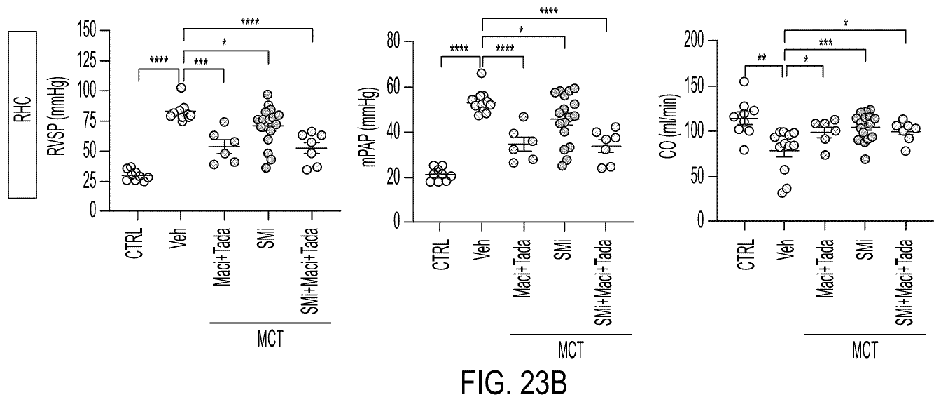

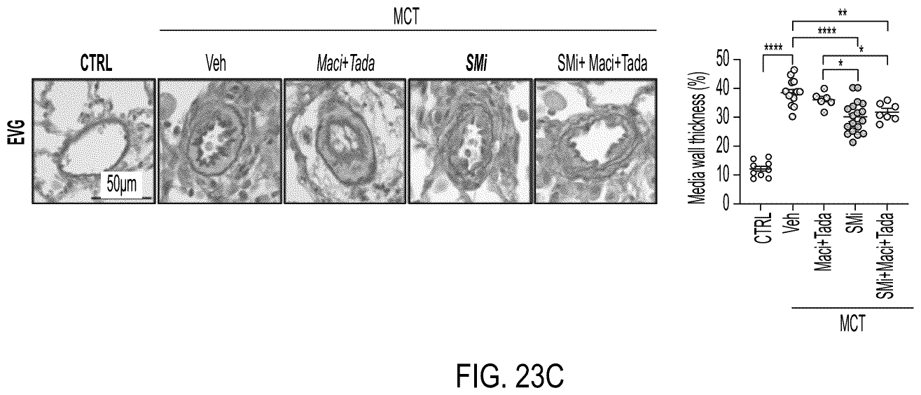

MCT (60 mg/kg) was administered by subcutaneous injection at day 0. Beginning in week 3, animals were exposed to vehicle or SMi treatment for 2 weeks. (FIG. 23A) Blood, RV and lung analysis was performed. Exemplary histology and tissue analysis demonstrated improved hypertrophy and right ventricle (RV) fibrosis with SMi alone or in combination with SoCs (FIG. 15A-15C). Right heart catheterization was performed and animals were assessed for RVSP (mmHg), mPAP (mmHg), and CO (ml/min) (FIG. 23B) As shown in FIG. 23C, MCT pulmonary hupertension animals treated with SMi alone or in combination with Maci (1 mg/kg) +Tada (10 mg/kg) showed improved vascular remodeling.

### Integrin α5β1 Inhibition Improves Cardiac Function in MCT Animals

To determine the selectivity of SMi for α5β1, MCT animals were prepared as described above and treated with SMi or a dual inhibitor of α5β1/αvβ1 (MRT).

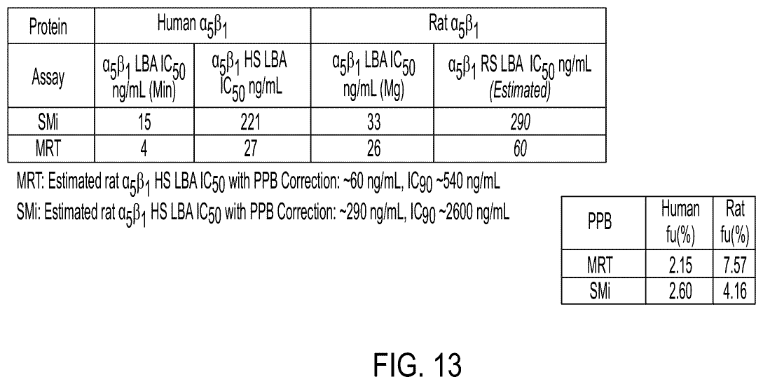

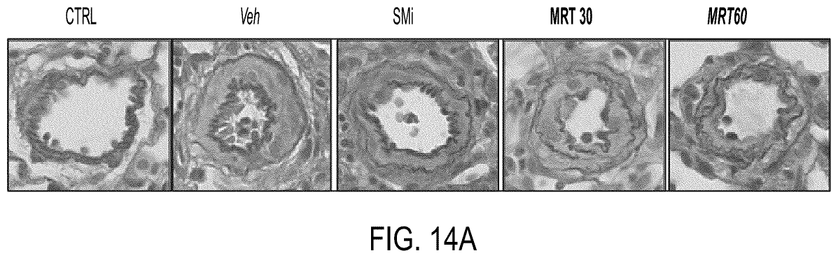

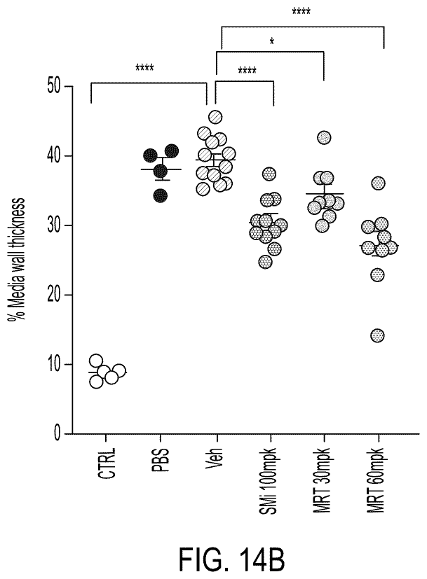

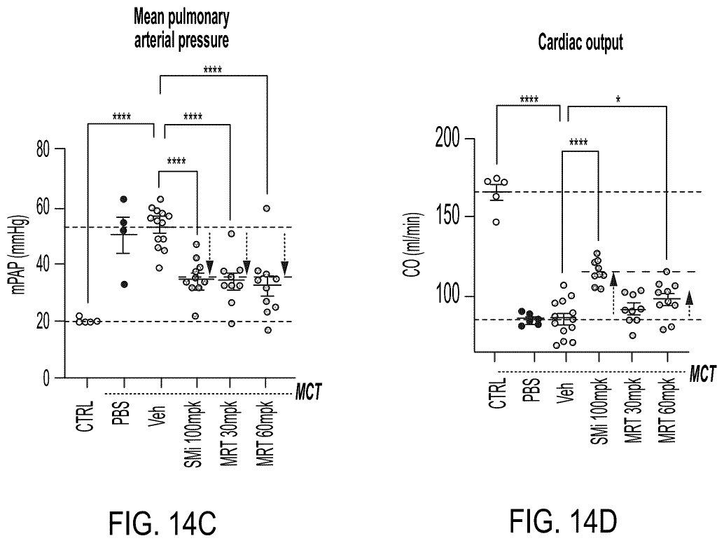

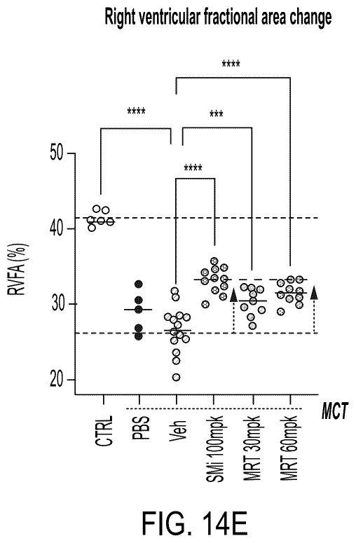

As shown in FIG. 14A-14E, both SMi and MRT treated MCT rats demonstrated improved cardiac function vascular remodeling demonstrated by EVG staining (FIG. 14A), media cell wall thickness (FIG. 14B), mean pulmonary arterial pressure (FIG. 14C), cardiac output (FIG. 14D) and right ventricular fractional area change (FIG. 14E). FIG. 13 shows exemplary pharmacokinetics of SMi and MRT. These data suggest that integrin α5β1 inhibition is responsible for the improvement of cardiac function in MCT animals.

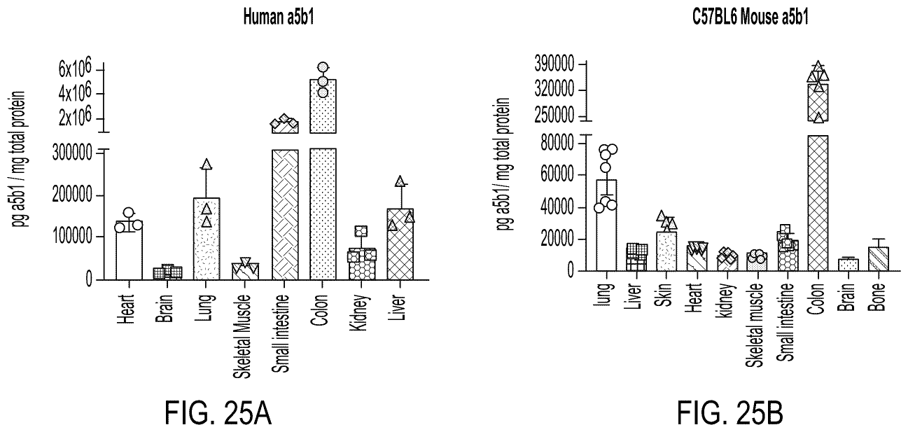

Protein levels of a5b1 integrin in human and mouse tissues were measured, confirming that a5b1 is highly expressed in human and mouse heart (FIG. 25A and FIG. 25B). In cultured cardiomyocytes stimulated by phenylephrine, an alpha-1 adrenergic receptor agonist, blocking a5b1 alleviated cardiomyocyte hypertrophy. Furthermore, in patient derived cardiac fibroblasts, a5b1 blockade reduced fibroblast activation. These experiments in in vitro systems further demonstrate the direct pharmacologic effects of a5b1 inhibition in cardiac cells.

### Integrin α5β1 Antibody Inhibitors in MCT Animals

MCT animals are prepared as described above and treated with integrin α5β1 antibody inhibitors. Anti-Integrin α5β1 antibody treated MCT rats are assessed for improved cardiac function vascular remodeling demonstrated by EVG staining, media cell wall thickness, mean pulmonary arterial pressure, cardiac output and right ventricular fractional area change. Cardiac function parameters are measured including improved cardiac output, stroke volume, media cell wall thickness, vascular remodeling, proliferation, and apoptosis are measured.

Example 4: In Vivo Efficacy of Integrin α5β1 Inhibitors in PAB

This example demonstrates efficacy of compound SMi in the right ventricle in Pulmonary Arterial Banding (PAB) rat model. Animals were divided into three study groups as follows:
(1) Control group/Sham (n=5)
(2) PAB+PBS vehicle orally twice a day (BID) (n=10)
(3) PAB+SMi at 100 mpk orally BID (n=10)

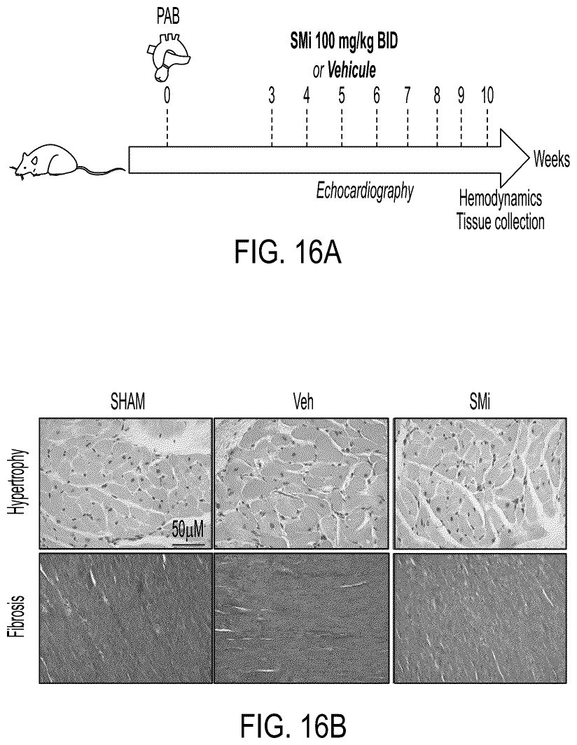

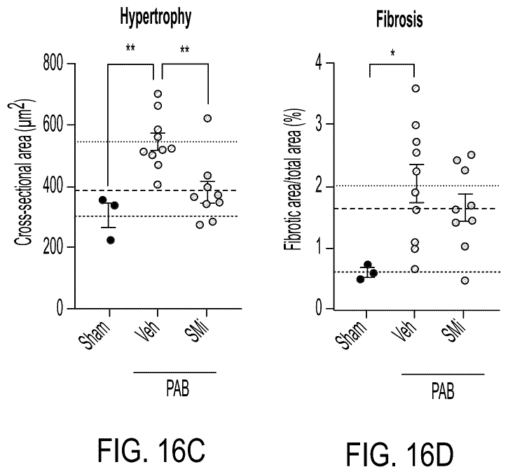

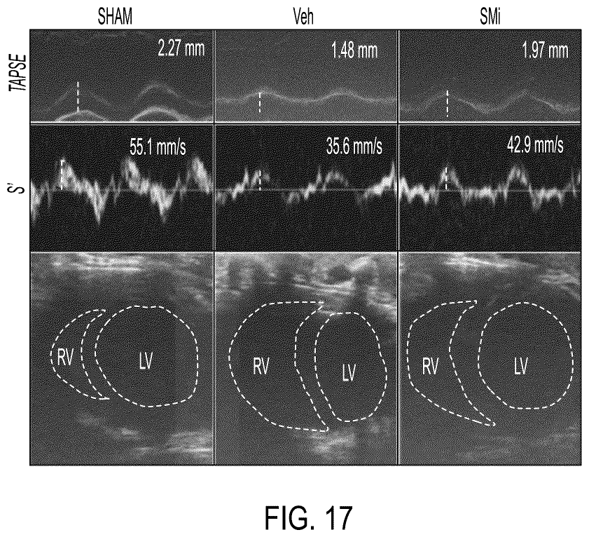

Pulmonary arterial constriction was surgically performed in rats at day 0. Beginning in week 4 following surgery, animals were exposed to vehicle or SMi treatment. Weekly echocardiography was performed from surgery to week 10. After a total of 10 weeks, animals were subjected to RV catheterization. Blood, RV and lung analysis was performed. exemplary histology and tissue analysis demonstrated improved hypertrophy and right ventricle (RV) fibrosis in Pulmonary Arterial banding (PAB) rat model treated with SMi (FIG. 16A-16D). Representative echocardiograph images of (PAB) rats treated with SMi are shown in FIG. 17.

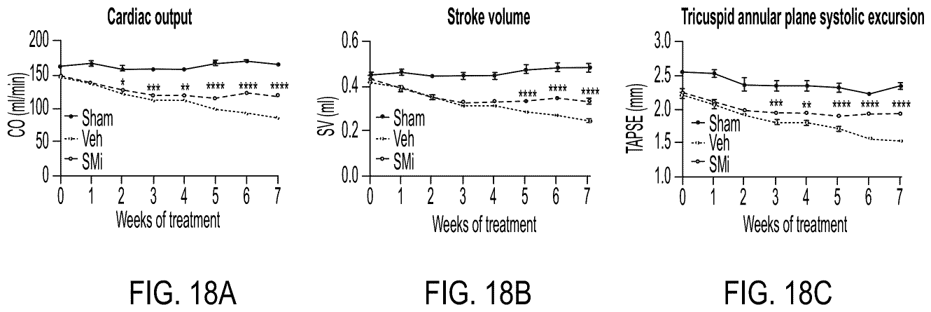

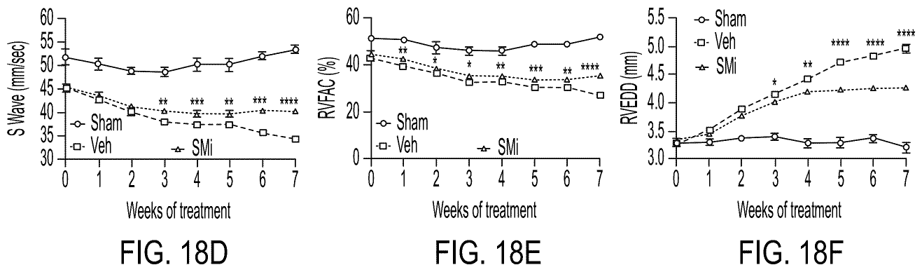

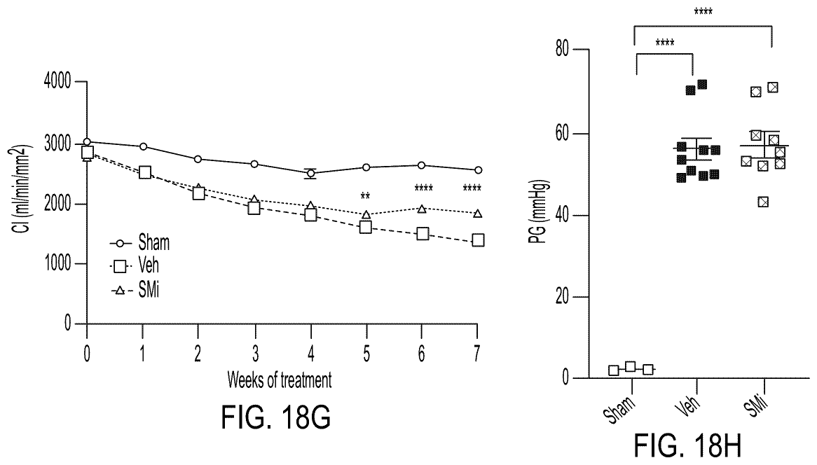

Hemodynamics and tissue collection analysis showed integrin α5β1 inhibition with SMi directly improved cardiac function over time compared to vehicle control in the RV PAB model. Cardiac output (FIG. 18A), stroke volume (FIG. 18B), Tricuspid annular plane systolic excursion (TAPSE) (FIG. 18C), S Wave (FIG. 18D), Right Ventricle Fractional Area Change (RVFAC) (FIG. 18E), RVEDD (FIG. 18F), cardiac index (CI) (FIG. 18G), pressure gradient (PG) (FIG. 18H).

### Right Heart Catheterization

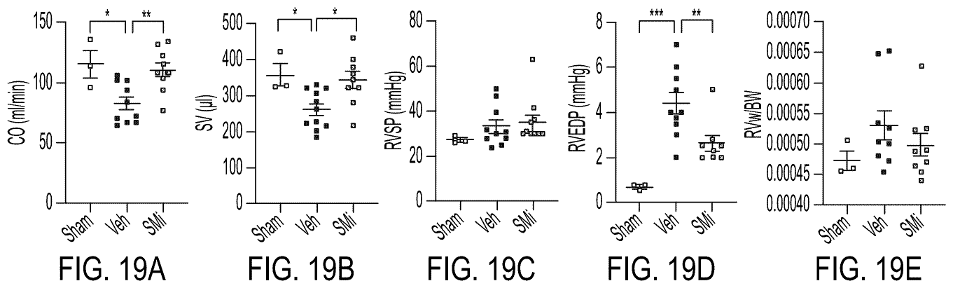

PAB rats were subjected to right heart catheterization (RHC) (using standard techniques and while the subject is in steady state). Exemplary cardiac parameters of Right Heart Catheterization in PAB rats treated with SMi are shown in FIG. 19A-19E. Treatment with integrin α5β1 inhibitor, SMi improved cardiac output, stroke volume, RVSP and RVEPD.

### Integrin α5β1 Antibody Inhibitors in PAB Animals

PAB rats are prepared as described above and treated with integrin α5β1 antibody inhibitors. Anti-Integrin α5β1 antibody treated PAB rats are assessed for improved cardiac function vascular remodeling demonstrated by EVG staining, media cell wall thickness, mean pulmonary arterial pressure, cardiac output and right ventricular fractional area change. Cardiac function parameters are measured including improved cardiac output, stroke volume, media cell wall thickness, vascular remodeling, proliferation, and apoptosis are measured.

### Example 5: Characterization of Integrin α5β1 Inhibitors

This example demonstrates methods for screening and characterization of integrin α5β1 inhibitors.

### Cell Adhesion Assay (CAA)

A 96-well assay plate was coated with 0.625 μg/ml purified recombinant human fibronectin consisting of domains 9 and 10, fused to glutathione S-transferase, and subsequently blocked with 1% bovine serum albumin. Cells expressing rat α5β1 were incubated with test samples, which were prepared in a serial dilution series, in a 96-well, deep well plate. 0.1 ml of the assay mixture consisted of 100,000 cells, the antibody samples, 50 mM HEPES pH 7.3, 150 mM sodium chloride, 1 mM magnesium chloride, 1 mM calcium chloride, 1% bovine serum albumin, and 10 mM glucose. After a pre-incubation time of 15 minutes, 0.1 ml of the assay mixture was transferred to the 96-well assay plates and the plate was incubated for 1 hour at room temperature. Non-adherent cells were removed using the Blue Washer (Blue Cat Bio) instrument and the amount of adherent cells was quantified using CellTiter Glo (Promega). Concentration-response curves were analyzed for IC50 values using 4-parameter non-linear regression analysis.

As shown in Table 3, a5b1 antibody inhibitors (e.g., P1D6 and M200) demonstrate nanomolar range affinity in blocking cell adhesion.

TABLE 3

| | P1D6 | M200 |
|---|---|---|
| IC50 (nM) | 5.6 | 6.8 |
| IC50 (ug/ml) | 0.84 | 1 |

### Solid Phase (SP) Assay

Solid Phase (SP) assays were used to measure antibody activity through binding competition with purified fibronectin isolated from rat plasma. The 384-well assay plate was coated with 2 μg/ml fibronectin and subsequently blocked with 1% bovine serum albumin. In a separate 384-well plate, 2 nM His-tagged rat α5β1 protein was incubated with the test sample in 50 mM HEPES pH 7.3, 150 mM sodium chloride, 0.5% bovine serum albumin, 1 mM magnesium chloride, 1 mM calcium chloride, and 0.05% Tween 20 for 1 hour at room temperature. 20 μl of the assay mixture was transferred to the fibronectin-coated assay plates and incubated 1 hour at room temperature. Unbound components were removed by 3 rounds of washing using the BioTek 405/TS plate washer. 20 μl of anti-6X Histag antibody conjugated to horse radish peroxide was added, incubated for 1 hour at room temperature, and unbound antibody was removed by 3 rounds of washing. The amount of His-tagged rat α5β1 protein bound to the anti-6X Histag antibody was quantified using Quantablue Fluorogenic substrate (Thermo Fisher) and concentration-response curves were analyzed for IC50 values using 4-parameter non-linear regression analysis.

### Ligand Binding Assay (LBA)

To measure the potency of samples against α5β1 in the cell-based ligand binding assay (LBA), cells expressing rat α5β1 were incubated with the test samples in a volume of 10 μl at room temperature for 15 minutes in buffer containing 50 mM HEPES pH 7.3, 150 mM sodium chloride, 1% bovine serum albumin, 2 mM magnesium chloride,2 mM calcium chloride, 15 mM glucose, 1.5% dimethyl sulfoxide, and 0.025% e780 fixable viability dye. 5 μl of 75 nM fibronectin isolated from human plasma and fluorescently labeled with Dylight 650 in 50 mM HEPES pH 7.3, 150 mM sodium chloride, and 1% bovine serum albumin was added to the cells. The samples were incubated for 45 minutes at room temperature, fixed with 0.8% formaldehyde for 30 minutes at room temperature, and washed with 50 mM Tris pH 7.5, 150 mM NaCl, 1 mM EDTA, and 1% bovine serum albumin. Fluorescence intensity for each cell was measured via flow cytometry. Dead cells were excluded from further analysis based on staining with the 780 fixable viability dye. Median fluorescence intensity for Dylight 650 was determined for each sample and concentration-response curves were analyzed for IC50 values using 4-parameter non-linear regression analysis.

TABLE 4
Ligand Binding of integrin α5β1 antibody inhibitors

| | LBA | | CAA | | SP | Biocare | |
|---|---|---|---|---|---|---|---|
| | IC50 | LBA | IC50 | CAA | IC50 | KD | Sequence |
| ID | (nM) | Emax | (nM) | Emax | (nM) | (nM) | group |
| mAb002 | 5.24 | 99% | 1.52 | 99.90% | 4.6 | N/D | Group 1 |
| mAb003 | 5.36 | 99% | 1.14 | 99.40% | 8.5 | 39.7 | Group 1 |
| mAb004 | 4.46 | 97% | 1.02 | 96.60% | 6.3 | 3.21 | Group 2 |
| mAb005 | 5.13 | 97% | 0.89 | 95.60% | 9.8 | 7.49 | Group 2 |
| mAb006 | 4.96 | 97% | 1.34 | 96.70% | 10.2 | 8.23 | Group 2 |
| mAb007 | 5.18 | 99% | 1.48 | 98.5% | 7.0 | 27.9 | Group 1 |
| mAb008 | 4.18 | 98% | 0.94 | 94.70% | 6.1 | 6.84 | Group 2 |
| mAb009 | 4.11 | 98% | 0.98 | 96.00% | 11.1 | 9.07 | Group 2 |

### Enumerated Embodiments

Embodiment 1: A method of treating pulmonary arterial hypertension (PAH) in a subject, comprising administering an integrin α5β1 inhibitor.

Embodiment 2: A method of treating a disease associated with increased expression or activity of integrin α5β1, comprising administering an integrin α5β1 inhibitor.

Embodiment 3: A method of treating a heart or lung disease, comprising administering an integrin α5β1 inhibitor.

Embodiment 4: The method of embodiments 1-3, wherein the disease is pulmonary hypertension WHO Group 1 pulmonary hypertension or pulmonary arterial hypertension (PAH), WHO Group 2 pulmonary hypertension, WHO Group 3 pulmonary hypertension, WHO Group 4 pulmonary hypertension, WHO Group 5 pulmonary hypertension, WHO Class I pulmonary hypertension or pulmonary arterial hypertension (PAH), WHO Class II pulmonary hypertension, WHO Class III pulmonary hypertension, WHO Class IV pulmonary hypertension.

Embodiment 5: The method of embodiments 1-3, The method of claim 3, wherein the disease is cardiac fibrosis, heart failure or right ventricle failure.

Embodiment 6: The method of embodiment 5, wherein the disease is cardiac fibrosis.

Embodiment 7: The method of embodiment 5, wherein the disease is heart failure.

Embodiment 8: The method of embodiment 5, wherein the disease is right ventricle failure.

Embodiment 9: The method of embodiment 3, wherein the disease is a heart disease.

Embodiment 10: The method of embodiment 3, wherein the disease is a lung disease.

Embodiment 11: The method of any one of the preceding embodiments, wherein the integrin α5β1 inhibitor is a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof.

Embodiment 12: The method of any one of the preceding embodiments, wherein the integrin α5β1 inhibitor is an antibody.

Embodiment 13: The method of any one of the preceding embodiments, wherein the integrin α5β1 inhibitor is an antibody that specifically binds integrin α5.

Embodiment 14: The method of any one of embodiments 1-12, wherein the integrin α5β1 inhibitor is an antibody that specifically binds integrin β1.

Embodiment 15: The method of any one of the preceding embodiments, wherein the integrin α5β1 inhibitor is an antibody that specifically binds integrin α5β1.

Embodiment 16: The method of embodiment 15, wherein the antibody is an integrin α5β1 antibody selected from the group consisting of volociximab (M200), PF-04605412 and MINT1526A.

Embodiment 17: The method of embodiment 16, wherein the antibody is volociximab (M200).

Embodiment 18: The method of embodiment 16, wherein the antibody is PF-04605412.

Embodiment 19: The method of embodiment 16, wherein the antibody is MINT1526A.

Embodiment 20: The method of embodiment 11, wherein the integrin α5β1 inhibitor is an integrin α5β1 antibody that competes for integrin binding with an antibody selected from the group consisting of volociximab (M200), P1D6, PF-04605412, MINT1526A, BMA5, BMB5, BMC5, HA5, JBS5, LS-C509074, LS-C24758, 1D9, 22B5, 24C7, 2D2, 3C2.2A8, 3C5, 5B11, MOR04055, MOR04624, P8D4, MOR04974, MOR04977, SG/19 and 18C12.

Embodiment 21: The method of any one of embodiments 1-10, wherein the integrin α5β1 inhibitor is a small molecule compound that binds integrin α5β1.

Embodiment 22: The method of any one of embodiments 1-10, wherein the integrin α5β1 inhibitor is a small molecule compound that specifically binds integrin α5.

Embodiment 23: The method of any one of embodiments 1-10, wherein the integrin α5β1 inhibitor is a small molecule compound that specifically binds integrin β1.

Embodiment 24: The method of any one of embodiments 1-10, wherein the integrin α5β1 inhibitor is a small molecule compound that specifically binds integrin α5β1.

Embodiment 25: The method of any one of embodiments 1-10, wherein the integrin α5β1 inhibitor is a compound of Formula (I) or a pharmaceutically acceptable salt thereof,

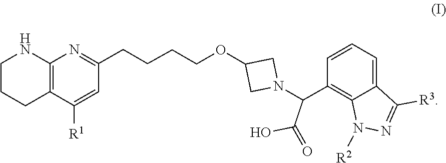

wherein:
R¹ is hydrogen or OMe;
R² is C₁₋₄alkyl optionally substituted with C₁₋₄alkoxy; and
R³ is C₁₋₄alkyl or C₃₋₅cycloalkyl.

Embodiment 26: The method of embodiment 25, wherein R2 is methyl or ethyl.

Embodiment 27: The method of embodiment 25 or 26, wherein R3 is methyl, ethyl, isopropyl, or cyclopropyl.

Embodiment 28: The method of any one of embodiments 25-27, wherein R1 is hydrogen.

Embodiment 29: The method of embodiment 25, wherein the compound of Formula (I) is selected from:

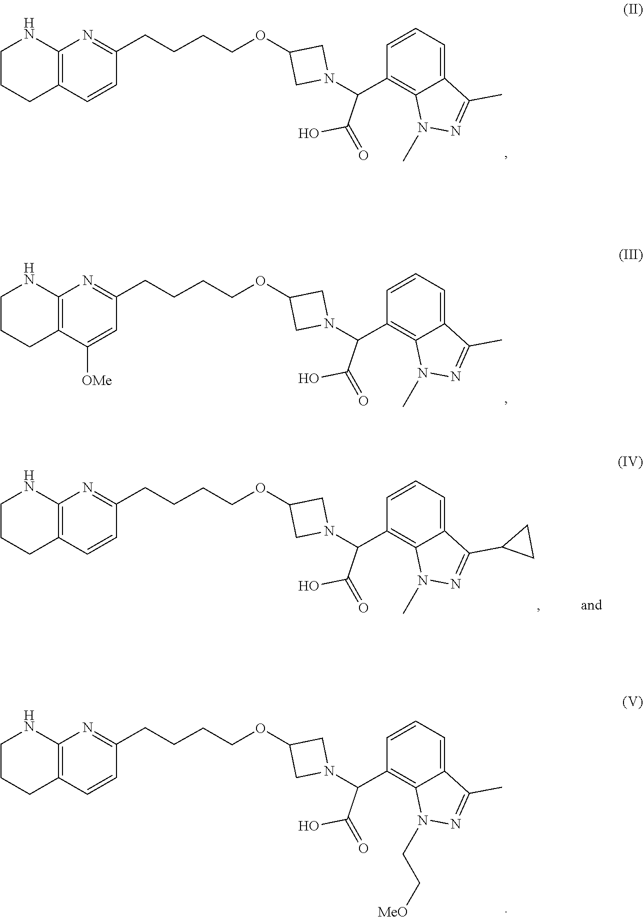 [SMILES: Cc1nn(C)c2c(C(C(=O)O)N3CC(OCCCCc4ccc5c(n4)NCCC5)C3)cccc12 ; COc1cc(CCCCOC2CN(C(C(=O)O)c3cccc4c(C)nn(C)c34)C2)nc2c1CCCN2 ; Cn1nc(C2CC2)c2cccc(C(C(=O)O)N3CC(OCCCCc4ccc5c(n4)NCCC5)C3)c21 ; COCCn1nc(C)c2cccc(C(C(=O)O)N3CC(OCCCCc4ccc5c(n4)NCCC5)C3)c21]

Embodiment 30: The method of any one of the preceding embodiments, wherein the integrin α5−1 inhibitor is administered orally, intravenously, subcutaneously, intranasally, transdermally, intraperitoneally, intramuscularly, or intrapulmonarily.

Embodiment 31: The method of any one of the preceding embodiments, further comprising administering to the subject a second therapy.

Embodiment 32: The method of any one of the preceding embodiments, wherein the second therapy is selected from the group consisting of anticoagulants, diuretics, a digitalis glycoside, calcium channel blockers, endothelin receptor antagonists, phosphodiesterase 5 (PDE5) inhibitors, prostanoids, prostanoids receptor agonists, soluble guanylate cyclase stimulators, and/or surgery.

Embodiment 33: The method of embodiment 32, wherein the second therapy is a prostanoid.

Embodiment 34: The method of embodiment 33, wherein the prostanoid is epoprostenol or treprostinil.

Embodiment 35: The method of embodiment 32, wherein the second therapy is an endothelin receptor antagonist.

Embodiment 36: The method of embodiment 35, wherein endothelin receptor is bosentan, ambrisentan, macitentan.

Embodiment 37: The method of embodiment 32, wherein the second therapy is a phosphodiesterase type-5 (PDE-5) inhibitor.

Embodiment 38: The method of embodiment 37, wherein the phosphodiesterase type-5 (PDE-5) inhibitor is sildenafil or tadalafil.

Embodiment 39: The method of embodiment 32, wherein the second therapy is a soluble guanylate cyclase (sGC) stimulator.

Embodiment 40: The method of embodiment 39, wherein the soluble guanylate cyclase (sGC) stimulator is riociguat.

Embodiment 41: The method of embodiment 31 or 32, wherein the second therapy is oxygen, Warfarin, furosemide, bumetanide, bendroflumethiazide, metolazone, spironolactone, amiloride, Digoxin, nifedipine, diltiazem, nicardipine, amlodipine, ambrisentan, bosentan, macitentan, sildenafil, tadalafil, epoprostenol, iloprost, treprostinil, riociguat, selexipag, surgery, pulmonary endarterectomy, and/or atrial septostomy.

Embodiment 42: The method of embodiment 31, wherein the second therapy is macitentan and/or tadalafil.

Embodiment 43: The method of any one of the preceding embodiments, wherein the subject has previously received a pulmonary hypertension therapy.

Embodiment 44: The method of embodiment 43, wherein the previously received pulmonary hypertension therapy is selected from the group consisting of anticoagulants, diuretics, a digitalis glycoside, calcium channel blockers, endothelin receptor antagonists, phosphodiesterase 5 (PDE5) inhibitors, prostanoids, prostanoids receptor agonists, soluble guanylate cyclase stimulators, and/or surgery.

Embodiment 45: The method of any one of the preceding embodiments, wherein administering the integrin α5β1 inhibitor reduces proliferation and/or survival of PASMCs, fibroblasts, right ventricle fibroblasts (RVFbs), vascular fibroblasts, adventitial fibroblasts, cardiomyocytes, and/or endothelial cells.

Embodiment 46: The method of any one of the preceding embodiments, wherein administering the integrin α5β1 inhibitor results in improved mean pulmonary arterial pressure (mPAP).

Embodiment 47: The method of any one of the preceding embodiments, wherein administering the integrin α5β1 inhibitor results in improved pulmonary vascular resistance (PVR).

Embodiment 48: The method of any one of the preceding embodiments, wherein administering the integrin α5β1 inhibitor results in improved systemic vascular resistance (SVR).

Embodiment 49: The method of any one of the preceding embodiments, wherein administering the integrin α5β1 inhibitor results in improved right atrial pressure (RAP).

Embodiment 50: The method of any one of the preceding embodiments, wherein administering the integrin α5β1 inhibitor results in improved cardiac output (CO).

Embodiment 51: The method of any one of the preceding embodiments, wherein administering the integrin α5β1 inhibitor results in improved heart rate (HR).

Embodiment 52: The method of any one of the preceding embodiments, wherein the α5β1 inhibitor is administered to improve exercise ability and delay clinical worsening of the disease.

Embodiment 53: The method of embodiment any one of the preceding embodiments, wherein the α5β1 inhibitor and the second therapy is administered is administered to improve exercise ability and delay clinical worsening of the disease.

Embodiment 54: The method of any one of the preceding embodiments, comprising administering the integrin α5β1 inhibitor to modulate the level of a biomarker in the subject, wherein the biomarker is brain natriuretic peptide (BNP) or N-terminal fragment (NT) of pro-BNP (NT-proBNP), TNFα, IFNγ, IL-6, IL-8, or IL-10.

Embodiment 55: The method of any one of the preceding embodiments, further comprising administering the integrin α5β1 inhibitor at a dose of 1 mg to 1000 mg.

Embodiment 56: The method of any one of the preceding embodiments, wherein the integrin α5β1 inhibitor is administered daily.

Embodiment 57: An integrin α5β1 inhibitor for use in treating pulmonary arterial hypertension (PAH) in a subject in need of treatment thereof, comprising administering the integrin α5β1 inhibitor and a pharmaceutical excipient to the subject.

### Equivalents and Scope

Those skilled in the art will recognize or be able to ascertain using no more than routine experimentation, many equivalents to the specific embodiments of the invention described herein. The scope of the present invention is not intended to be limited to the above Description, but rather is as set forth in the following claims.

1. A method of treating pulmonary arterial hypertension (PAH) in a subject, comprising administering an integrin α5β1 inhibitor.

2. A method of treating a heart or lung disease in a subject, comprising administering an integrin α5β1 inhibitor.

3. The method of claim 2, wherein the disease is pulmonary hypertension WHO Group 1 pulmonary hypertension or pulmonary arterial hypertension (PAH), WHO Group 2 pulmonary hypertension, WHO Group 3 pulmonary hypertension, WHO Group 4 pulmonary hypertension, WHO Group 5 pulmonary hypertension, WHO Class I pulmonary hypertension or pulmonary arterial hypertension (PAH), WHO Class II pulmonary hypertension, WHO Class III pulmonary hypertension, WHO Class IV pulmonary hypertension.

4. The method of claim 2, wherein the disease is cardiac fibrosis, heart failure or right ventricle failure.

5. The method of any one of the preceding claims, wherein the integrin α5β1 inhibitor is a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof.

6. The method of claim 5, wherein the integrin α5β1 inhibitor is an integrin α5β1 antibody selected from the group consisting of volociximab (M200), PF-04605412 and MINT1526A.

7. The method of claim 5, wherein the integrin α5β1 inhibitor is an integrin α5β1 antibody that competes for integrin binding with an antibody selected from the group consisting of volociximab (M200), P1D6, PF-04605412, MINT1526A, BMA5, BMB5, BMC5, HA5, JBS5, LS-C509074, LS-C24758, 1D9, 22B5, 24C7, 2D2, 3C2.2A8, 3C5, 5B11, MOR04055, MOR04624, P8D4, MOR04974, MOR04977, SG/19, and 18C12.

8. The method of any one of claims 1-4, wherein the integrin α5β1 inhibitor is a small molecule compound that binds integrin α5β1.

9. The method of claim 8, wherein the integrin α5β1 inhibitor is a compound of Formula (I) or a pharmaceutically acceptable salt thereof, 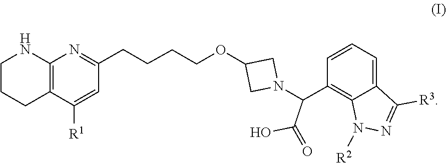
wherein:
R¹ is hydrogen or OMe;
R² is C₁₋₄alkyl optionally substituted with C₁₋₄alkoxy; and
R³ is C₁₋₄alkyl or C₃₋₅cycloalkyl.

10. The method of claim 9, wherein R2 is methyl or ethyl.

11. The method of claim 9 or 10, wherein R3 is methyl, ethyl, isopropyl, or cyclopropyl.

12. The method of claim 9, wherein the compound of Formula (I) is selected from:

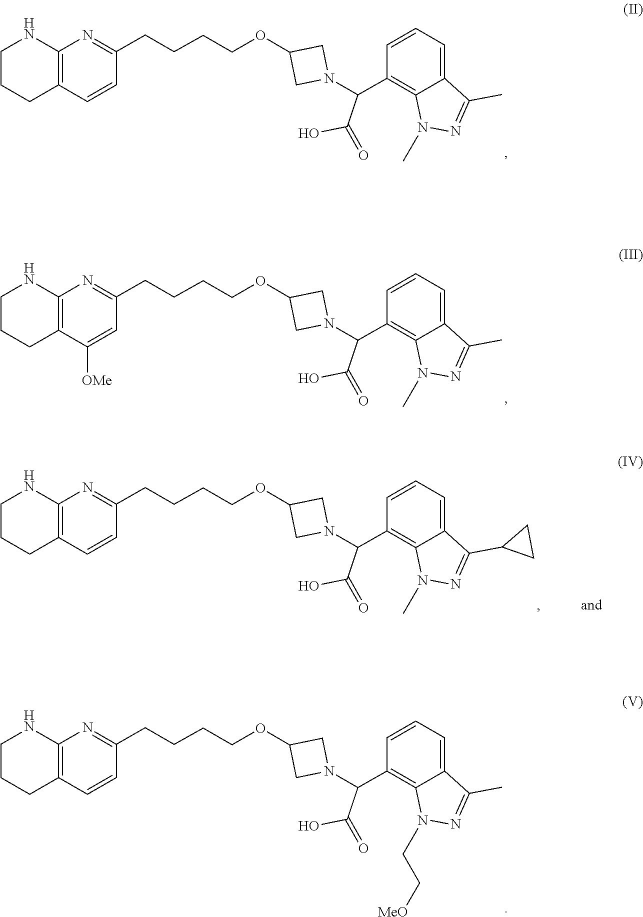 [SMILES: Cc1nn(C)c2c(C(C(=O)O)N3CC(OCCCCc4ccc5c(n4)NCCC5)C3)cccc12 ; COc1cc(CCCCOC2CN(C(C(=O)O)c3cccc4c(C)nn(C)c34)C2)nc2c1CCCN2 ; Cn1nc(C2CC2)c2cccc(C(C(=O)O)N3CC(OCCCCc4ccc5c(n4)NCCC5)C3)c21 ; COCCn1nc(C)c2cccc(C(C(=O)O)N3CC(OCCCCc4ccc5c(n4)NCCC5)C3)c21]

13. The method of any one of the preceding claims wherein the integrin α5β1 inhibitor is administered orally, intravenously, subcutaneously, intranasally, transdermally, intraperitoneally, intramuscularly, or intrapulmonarily.

14. The method of any one of claims 1-13, further comprising administering to the subject a second therapy.

15. The method of claim 14, wherein the second therapy is selected from the group consisting of anticoagulants, diuretics, a digitalis glycoside, calcium channel blockers, endothelin receptor antagonists, phosphodiesterase 5 (PDE5) inhibitors, prostanoids, prostanoids receptor agonists, soluble guanylate cyclase stimulators, and/or surgery.

16. The method of claim 15, wherein the second therapy is oxygen, Warfarin, furosemide, bumetanide, bendroflumethiazide, metolazone, spironolactone, amiloride, Digoxin, nifedipine, diltiazem, nicardipine, amlodipine, ambrisentan, bosentan, macitentan, sildenafil, tadalafil, epoprostenol, iloprost, treprostinil, riociguat, selexipag, surgery, pulmonary endarterectomy, and/or atrial septostomy.

17. The method of any one of the preceding claims, wherein administering the integrin α5β1 inhibitor reduces proliferation and/or survival of PASMCs, fibroblasts, right ventricle fibroblasts (RVFbs), vascular fibroblasts, adventitial fibroblasts, cardiomyocytes, and/or endothelial cells.

18. The method of any one of the preceding claims, comprising administering the integrin α5β1 inhibitor to modulate the level of a biomarker in the subject, wherein the biomarker is brain natriuretic peptide (BNP) or N-terminal fragment (NT) of pro-BNP (NT-proBNP), TNFα, IFNγ, IL-6, IL-8, or IL-10.

19. The method of any one of the preceding claims, comprising administering the integrin α5β1 inhibitor at a dose of 1 mg to 1000 mg.

20. An integrin α5β1 inhibitor for use in treating pulmonary arterial hypertension (PAH) in a subject in need of treatment thereof, comprising administering the integrin α5β1 inhibitor and a pharmaceutical excipient to the subject.
